(19) United States
(12) Patent Application Publication
(10) Pub. No.: US 2003/0153023 A1
(43) Pub. Date: Aug. 14, 2003

(54) Enumeration method of analyte detection

(76) Inventors: Timothy W. Starzl, Boulder, CO (US); Scott M. Clark, Denver, CO (US); Mary Beth Vellequette, Louisville, CO (US); H. John Hanlin, Louisville, CO (US); Steven W. Metzger, Fort Collins, CO (US)

Correspondence Address: SHERIDAN ROSS PC, 1560 BROADWAY, SUITE 1200, DENVER, CO, 80202

(21) Appl. No.: 10/084,632

(22) Filed: Feb. 25, 2002

Related U.S. Application Data
(63) Continuation-in-part of application No. 09/311,663, filed on May 13, 1999, now abandoned.

(51) Int. Cl. G01N 33/53; G01N 33/537; G01N 33/543; G06F 19/00; G06K 9/00

(52) U.S. Cl. 435/7.92; 382/128; 702/19

(57) ABSTRACT

This invention is directed to an optically-based method and system for analyte detection using solid phase immobilization, specific analyte labels adapted for signal generation and corresponding processes for the utilization thereof. The enumeration detection method disclosed herein narrows the area for signal observation, thus, improving detectable signal to background ratio. The system is comprised of a platform/support for immobilizing a sample stage having a labeled sample (analyte complex) bound thereto, a radiation source, an optical apparatus for generating and directing radiation at said sample and a control that obtains data and then conducts analyses using digital image data. Upon engagement of the system, the sample generates a signal capable of differentiation from background signal, both of which are collected and imaged with a signal detector that generated a sample image to a data processing apparatus. This apparatus receives signal measurements and, in turn, enumerates individual binding events. Generated signal may be increased via selected mass enhancement. The invention, enumeration assay methodology detecting individual binding events, may be used, for example, in analyses to detect analyte or confirm results in both research, commercial and point of care applications.

This is a continuation-in-part (CIP) application of application Ser. No. 09/311,663 having a filing date of May 13, 1999.

### FIELD OF THE INVENTION

This invention relates to the general fields of molecular biology, biochemistry, microbiology and biological research, specifically, to detection of analytes, and more specifically, to an enumeration assay method and system for the detection of individual binding events. The present invention enables the detection of low concentrations of individual binding events. The present invention enables the detection of low concentrations of specific molecules of interest (analytes) using solid phase immobilization and optical signals capable of generating, detecting and measuring mass changes.

### BACKGROUND AND PRIOR ART

Improving the lower limit of detection—the threshold of detection of chemical sensitivity—has been a primary objective of ligand binding assay development since its inception. It has long been recognized that optical detection methods defined by the relationship between various optical interactions with mass on a solid phase, in particular ellipsometry, are capable in principle of providing a high level of sensitivity for standard binding reactions when compared to alternative signal generation methods, for example, enzyme/substrate interaction, fluorescent emission, radioactive emission and color emission. It has also been recognized that mass could be added to the binding complex in order to amplify the optical signal generated. It has been demonstrated that large amounts of mass can be successfully conjugated to the binding complex to this end. An example of this method is provided by the optical ellipsometric immunoassay (OpTest™, DDx, Inc.), a detection system for molecular and microscopic scale events, that measures interactions between biological samples and light.

The prior art discloses several imaging methods for the detection of analytes. U.S. Pat. No. 5,599,668 to Stimpson et at., entitled Light Scattering Optical Waveguide Method for Detecting Specific Binding Events, discloses a DNA-hybridization imager that detects the scattering of light directed into a waveguide, using labeled microspheres (beads) and visually monitors binding by video imaging. The waveguide device is required as a solid phase and imaging is achieved with a CCD camera and frame grabber software.

Allen et al., U.S. Pat. No. 5,488,567, entitled Digital Analyte Detection System is directed to the digital detection of the presence of analyte particles based upon illumination thereof. Distinct pixel regions of the sample are illuminated and the emitted signal detected.

A novel optical biosensor system is taught in A Biosensor Concept Based on Imaging Ellipsometry for Visualization of Biomolecular Interactions (Jin et al. (1995) Anal. Biochem. 232:69. The biosensor system utilizes specificities of biomolecular interactions in combination with protein patterned surfaces and imaging ellipsometry and a CCD camera to collect data.

The general use of imaging ellipsometry in conjunction with a CCD camera and framegrabber board is disclosed in Performance of a Microscopic Imaging Ellipsometer (Beaglehole (1988) Rev. Sci. Instrum. 59(12):2557. No type of life science or biological system application of the imaging is suggested.

A Method for Detecting the Presence of Antibodies using Gold-Labeled Antibodies and Test Kit are taught in U.S. Pat. No. 5,079,172 to Hari et al. This methodology is directed to detecting labeled microparticles using microscopy, for example, an electron microscope imaging system.

Chemical and biochemical analysis involving the detection and quantitization of light occurs in a variety of situations. One application is the detection of analytes for the determination of the presence or amount of a particular analyte. In many assays for analytes, the concern lies with either absorption or emission of light radiation (e.g., fluorescence or chemiluminescence). In such cases, a sample is irradiated and the effect of the sample on the transmitted or emitted light is detected. In the case of emitted light resulting from irradiation, non-analyte molecules may also emit light creating relatively high background noise and resulting in the introduction of substantial error in measurement. Additional systematic errors may also collectively contribute to the noise associated with measurement.

The quality of chemical measurements involving light can be defined in terms of the ratio of a suitable measurement of the optical signal from a sample due to the presence of analyte to the noise variation inherent within the system. The source of noise that may affect the results may come from anywhere within the optical path, including the sample, the signal source, detector variation and environmental interference. However, these variations are not necessarily inherent, and may also include externally imposed or induced variations. In general, efforts to augment this signal to noise (S/N) ratio have centered on improving the sensitivity of a measurement apparatus so as to reduce the “detection limit” associated with a particular analyte. The detection limit refers to the analyte concentration within a sample above which the signal attributable to the presence of analyte is such that a desired S/N ratio is achieved. In practice, this detection limit is ascertained by conducting an experimental procedure designed to elicit an optical signal related to analyte concentration. Specifically, data relating to signal and noise intensity is plotted in the form of a calibration curve for a range of analyte concentrations, thereby enabling straightforward determination of the detection limit.

The determination of concentration in unknown samples is then effected by comparing the signal obtained experimentally from the unknown with the calibration curve. A typical unit of concentration in chemical measurements is moles/liter [i.e., Molarity (M)], where a mole is defined as Avogadro's number (6.0225×10²³). Unfortunately, even the most sensitive conventional experimental techniques have detection limits on the order of about one femtomolar (fW), or nearly one billion analyte particles per liter.

Measurements in which concentration is determined by reference to a calibration curve may be characterized as being inherently “analog” rather than “digital”. That is, a signal correlated with analyte concentration is initially produced by the measurement device. The calibration curve is then consulted to obtain an approximation of the analyte concentration. Since the calibration curve is continuous as a function of concentration, the concentration derived from the calibration curve generally is not an integer. In contrast, digital measurement data are often embodied in binary (i.e., two-level) signals that unequivocally represent specific integers. Accordingly, a fundamental difference between analog and digital modes of measurement is that the addition of a single additional analyte to a sample analyzed using analog means cannot be unambiguously detected. Although dramatic improvements have been made in the accuracy of chemical measurements, such advancements have been based on the fundamentally analog concepts of increasing signal and reducing noise.

In molecular samples involving low levels of analyte concentration a digital measurement methodology affords at least two advantages: no calibration curve reference and detection of single molecules in a sample. Enumeration methodologies are useful in samples where the analyte concentration is sufficiently low that statistical noise accompanying each binary measurement value remains less than the difference between successive integers. Accordingly, it is an object of the present invention to provide an optical technique for determining low levels of analyte concentration by means of an intrinsically digital measurement scheme adapted for individual binding event detection.

To date, development in the prior art has been directed to imaging of an area of binding, as opposed to distinct video pixels (an array of digitized picture elements) or individual binding sites. The various problems of the prior art are overcome by the present invention. Shortcomings of the prior art include, for example, limitation to emission based reaction detection, averaging and/or detecting reactions over an area or plurality of pixels and the necessity of both signal producing and non-producing areas and distribution determination. The present invention overcomes these drawbacks by providing an integrated system and methodology for analyte detection through enumeration of individual binding events. While prior art is suitable for qualitative and limited quantitative determination, none of the prior art can be easily and efficiently used in the accurate enumeration of individual analyte binding events, nor does it teach the enhanced performance characteristics disclosed herein. The present invention provides improved enumeration sensitivity and accuracy, thereby obviating the herein-described prior art.

A prior art search failed to reveal any references disclosing the present invention or making it obvious to one of ordinary skill in the art. Furthermore, combinations of the disclosures of the referenced prior art would not teach the present invention nor would such a combination make the invention obvious. No reference teaches or suggests, the novel characteristics or combinations employed in the instant detection of solid-phase bound analyte on a molecule-by-molecule basis. The methods disclosed herein are useful, for example, for the solid phase detection of biological markers where the frequency, density or distribution of binding events is below the detectable threshold of conventional immunoassay, DNA probe and immuno-chromatographic detection methodologies.

### SUMMARY OF THE INVENTION

The instant invention is based on novel methods of analyte detection as a means for detection of specific molecules using solid phase immobilization and optical signal generation. More specifically, this invention comprises the use of optical signals and detectors capable of detecting and measuring mass changes resulting in analyte detection. This method further relates to commercial applications for automating detection and interfacing with existing assay methodologies, therefore lending itself to commercial applications, for example, high throughput pharmaceutical screening and point-of-care detection. That is, this invention is directed to the solid phase, optical detection and enumeration of individual binding events mediated by specific binding interactions.

This invention is defined by analyte solid phase immobilization, a signal generator, a signal carrier including optical pathways, a means of signal detection and novel data analysis. It encompasses a method for improving the delectability of individual binding events by utilizing a narrow optical beam size or by parsing or dividing a larger beam into smaller virtual beams using a diode array or a charged-coupled device (CCD) detector. The use of various optical signals and physical amplification elements is discussed herein.

In its broadest embodiment, the invention is directed to a method and system for solid phase, optical detection and enumeration of individual target analyte binding events comprising the steps of: immobilizing an analyte complex on a reflective or transmissive substrate directly from solution, said complex comprising a target analyte complexed with at least one signal generator element conjugated to at least one secondary analyte specific binding element; reflecting or transmitting electromagnetic radiation from or through the substrate having the analyte complex immobilized thereon; capturing a signal generated from said reflecting or transmitting of electromagnetic radiation; and, analyzing the signal for the presence and/or amount of analyte present.

More specifically, a system and method for digitally detecting the presence of analyte particles within a sample is disclosed herein. Each analyte complex is disposed to generate an optically detectable response upon stimulation (e.g., illumination) in a known manner. Furthermore, signal generators may be passive or active. Passive signal generators include those that interact with, but do not process, illumination, e.g., absorption, scattering. Active signal generators are those that actively transform photonic energy through a change in state, i.e., fluorescence, chemiluminescence and plasmon resonance. For stimulation or illumination, the digital analyte detection system includes optical apparatus for illuminating a multiplicity of distinct pixel regions within the sample so as to induce each of the analyte complexes included therein to generate an optical signal, i.e., photons. As discussed herein, Stimpson et al. and Allen et al. employ the use of CCDs and pixels for detection purposes. In the instant invention, the pixel regions are dimensioned such that the number of analyte complexes included within each region is sufficiently small that the aggregate optical signal generated by each region is less than a maximum detection threshold, preferably, 1 particle per pixel or multiple pixels per particle.

The digital detection system further includes apparatus for measuring the optical signal generated from each pixel region. A data processing network receives the optical signals, quantifies the signals, and based on the measurements, counts the number of analyte particles within each pixel region so as to determine the number of analyte particles within the sample.

The detection techniques of the present invention can be used for detecting a wide variety of analytes. As used herein, the term “optical response” is intended to collectively refer to the signal generation from a single analyte complex, however induced. In addition, the term “generated signal” as used herein corresponds to a measurement of the optical responses detected from a particular pixel or pixel region. The assay sample medium is preferably a solid phase bound analyte complex in which detectable label not bound to an analyte may be removed through conventional washing procedures.

In a preferred embodiment the analyte particles within each pixel region are measured individually based on discrete signal units providing optical responses substantially above a background noise level. The magnitude of each optical response is required to be large enough to allow the particular photodetection apparatus employed to discriminate between optical responses and ambient background noise. One or more optical responses of a signal unit may be associated with a single analyte particle, but the number of units will be substantially identical for each analyte particle. For the most part, the number of signal units per analyte complex will be more than one.

The assay sample medium often has low concentrations of analyte, generally at picomolar or less, frequently femtomolar or less. Assay volumes are usually less than about 100 μl, frequently less than 10 μl and may be 1 μl or less. It is desirable to match the CCD pixels to the signal generator label, preferably ranging in size from 5 nm to 5 microns, such that the labels can be individually detected. The actual size of the CCD pixels is irrelevant in that this is accomplished through magnifying optics.

Assays normally involve specific binding pairs, where by specific binding pairs it is intended that a molecule has a complementary molecule, where the binding of the elements of the specific binding pair is at a substantially higher affinity than random complex formation. The elements of a specific binding pair can be referred to as “ligands” and “receptors.” Generally receptors are immobilized to the solid phase to capture, or immobilize, the analyte of interest (the “ligand”) from a fluid sample. Thus, specific binding pairs may involve haptens and antigens (referred to as “ligands”) and their complementary binding elements, such as antibodies, enzymes, surface membrane protein receptors, lectins, etc. (generally known as “receptors”). Specific binding pairs may also include complementary nucleic acid sequences, both naturally occurring and synthetic, either RNA or DNA, where for convenience nucleic acids will be included within the concept of specific binding elements comprising ligands and receptors.

In carrying out the assay, a conjugate of a specific binding element and a detectable and discrete label is involved. Methods of preparing these conjugates are well known, and—are, therefore, not discussed herein. Depending upon the analyte, various protocols may be employed, which may be associated with commercially available reagents or such reagents which may be modified.

Other features and advantages of the instant invention will become apparent from the following detailed description, taken in conjunction with the accompanying figures, that illustrate by way of example, the principles of the instant invention.

### BRIEF DESCRIPTION OF THE DRAWINGS

FIG. 1 illustrates the determination of mass per unit volume or equivalent thereof in standard immunoassay methodology;

FIG. 2 depicts optical averaging occurring over an assay area;

FIG. 3 depicts the highly non-homogeneous assay area integration;

FIG. 4 illustrates the statical reduction to insignificance when low numbers of binding events are averaged over a large assay area;

FIG. 5 shows small beam ellipsometry or scatterometry provide higher relative signal for discreet binding events;

FIG. 6 illustrates the methodological approach for surface resolution, thereby approximating discreet binding event identification;

FIG. 7 illustrates laser determination of aggregate response;

FIG. 8 depicts scanning micro-laser configuration for the determination of individual cellular scale readings;

FIG. 9 illustrates relative size in relation to detection;

FIG. 10 depicts CCD and/or diode array beam employed to parse the laser beam into discrete signals;

FIG. 11 illustrates the variability of optical signals useful for detection and resolution purposes;

FIG. 12 shows examples of optical signal formats: past, current and prophetic;

FIG. 13 illustrates the scale of potential scanning micro-laser configurations;

FIG. 14 depicts optical enhancement potential;

FIG. 15 depicts the preferred instrumentation embodiment of the instant invention;

FIG. 16 illustrates a block diagram of an instrument in which the test piece is movable in X and Y directions;

FIG. 17 is a perspective view illustrating certain of the components of FIG. 16 including a laser subsystem, a X-Y subsystem, an optical subsystem and the light collection device;

FIG. 18 is an exploded view of the components of FIG. 17;

FIG. 19 illustrates some of the components of FIG. 18 assembled together but with laser subsystem and Z movement components being shown in exploded view;

FIG. 20 illustrates some of the components of FIG. 17 and diagrammatically depicts the light beam input from the laser subsystem and the light received by the optical subsystem to be input into the light collection device;

FIG. 21 illustrates a front panel of the instrument of FIG. 16 including controls related to controlling image data and indicators related to information associated with a number of subspots for one spot on the test piece;

FIG. 22 is a graph illustrating a histogram of the number of pixels at different grey levels;

FIG. 23 is a flow diagram related to the providing of instrument settings and positions;

FIG. 24 is a flow diagram related to main steps conducted in testing one or more subspots of one or more spots found on a test piece;

FIG. 25 is a flow diagram identifying certain major steps involved with processing of image data using light intensity; and

FIG. 26 is a flow diagram identifying certain major steps related to image analysis using size or appearance.

### DETAILED DESCRIPTION

It is understood that both the foregoing general description and the following detailed description are exemplary and explanatory only and are not restrictive of the invention as claimed. The general principles and conditions for analyte detection, manipulations (hybridization and amplification), and optics (lasers and ellipsometry) are well known in the art. The instant invention describes a novel method of detection for individual binding events.

One skilled in the art recognizes that the instant invention, as disclosed herein, may be performed in a broad range of samples. Such samples include, for example, biological samples derived from agriculture sources, bacterial and viral sources, and from human or other animal sources, as well as other samples such as waste or drinking water, agricultural products, processed foodstuff and air. The present invention is useful for the detection of low numbers of immobilized specific molecules.

The present invention is embodied in a method employing optical signals and detectors capable of detecting and measuring mass changes in a sample assay area. Regardless of the specific application of the instant invention, the methodology details are calculated according to protocols well known in the art, as well as those disclosed herein. Further, the refinement of said necessary calculations is routinely made by those of ordinary skill in the art and is within the ambit of tasks routinely performed by them without undue experimentation.

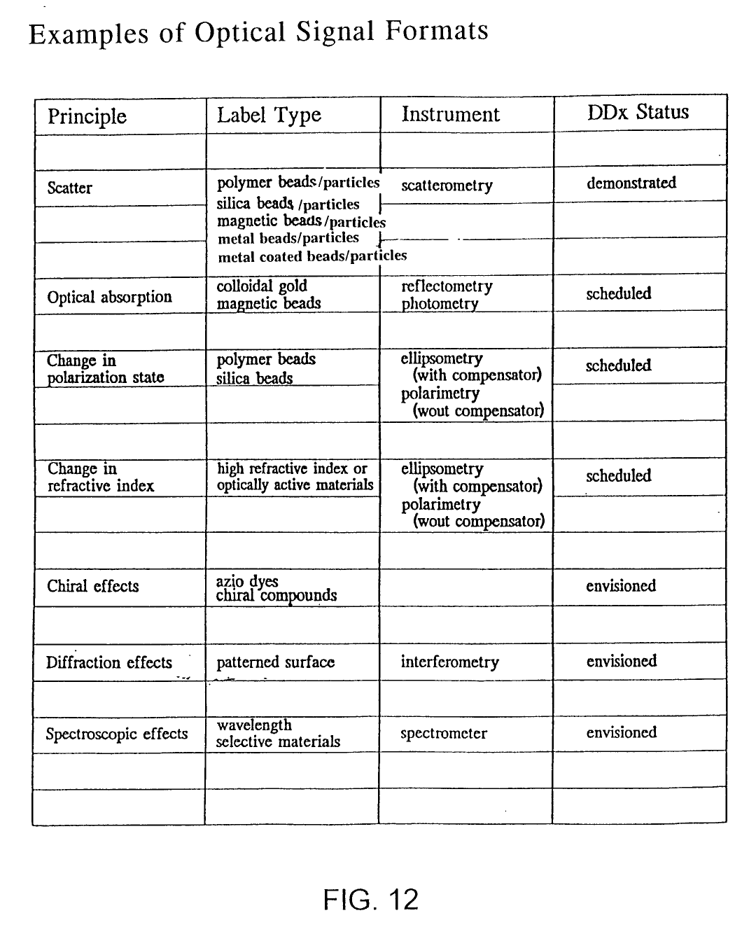

This application references and specifically discusses the use of ellipsometry as the optical method; this convention is for convenience only. It is understood that this methodology applies to a range of optical signal types, including those referenced in FIG. 12. It is specifically envisioned that the performance of a variety of optical methods will be substantially improved by adopting the general approach described herein. In particular, scattering methods form the basis of one class of instruments that is distinct from ellipsometry. Other effects such as absorption, refractive index change, and diffraction are used within an essentially similar optical configuration, and may provide particular result benefits. In application, the defining of the optical signal format drives the choice of appropriate immobilization surfaces and suitable data analysis methods for the purpose of distinguishing individual binding events. Thus, the attributes of the immobilization system and data analysis system are contingent upon the attributes of the selected optical signal format. The purpose of the optical signal format (the conjunction of a signal carrier, signal generator and signal detector) is to cause and detect a signal. The ability to distinguish the signal caused by the signal generator label from the signal caused by the background platform upon which the system is run, the solid phase, is fundamental to the optical signal format.

Definitions helpful in understanding the specification and claims are included throughout the instant disclosure. The definitions provided herein should be borne in mind when these terms are used in the following examples and throughout the instant application. The disclosures made herein are limited, for simplicity and convenience, to assays directed to the addition of mass (e.g. ligand binding assays), and reference is made to immunoassay methods. However, the same principles of optical signal detection generally apply to systems where mass is removed from the system (e.g. lytic or dissociation assays), and this invention is, thus, applicable to assays measuring mass change and derivatives thereof. Furthermore, this invention is directed to both transmission- and reflection-based solid phase assays.

Those skilled in the art readily recognize the present invention is broadly applicable in the areas of art described herein. The following examples and detailed descriptions serve to explain and illustrate the present invention. Said examples are not to be construed as limiting of the invention in anyway. Various modifications are possible within the scope of the invention.

The advent of small bead conjugation, beads ranging in diameter from 25 nm to 20 microns, opened the way to a new form of signal detection. That signal detection is described in the present application, and hereinafter referred to as the enumeration method. The instant invention enables the detection of individual binding events. The principle being to narrow the size, actual or virtual, of the area observed for signal, thereby improving the ratio of true signal to background signal, while concurrently using selected mass enhancement elements to increase the signal generated. Certain macromolecules or cellular bodies are large enough that they may be detected without additional mass enhancement, i.e., without secondary labels or reagents. The present invention, thus, solves the problem of detection of low concentrations of specific molecules of interest (analytes) using solid phase immobilization and optical signals capable of detecting and measuring mass changes.

In one embodiment, such mass changes are additively achieved or mediated by analyte complexing or binding via steric, shape mediated or other non-covalent, interactions with a ligand binding pair. Examples of such interactions include antigen-antibody binding, nucleic acid (DNA, RNA, PNA) binding, and other specific macromolecular (protein, glycoprotein, or carbohydrate binding) interactions. Alternatively, mass change is subtractively achieved through specific enzymatic, chemical or other specific dissociating or lytic agents. Examples of assay systems utilizing specific binding or lytic interactions suitable for mass change analysis include, for example, immunoassay, hybridization assay, protein binding assay and enzyme activity assay.

Alternate embodiments of this invention include secondary reagents used to amplify or differentiate the optical signal associated with the binding or lytic event through specific enhancement or alteration of that signal. Such enhancement involves the addition of simple mass to a completing event, or the generation of a differentiable type of signal from a specific species or process. Alternatively, such enhancement involves the alteration of one or more of the elements of the binding or lytic event generating a differentiable optical signal, or the enhancement initiates a detectable self-assembly or aggregation process.

In solid phase assay of the type described herein, results are typically derived from a statistical distinction between the assay signal and the background noise. This type of assay is typically performed utilizing macro-scale volumes (>1 μl) of a liquid sample or suspension. Similarly, the immobilization area typically used for this type of assay is also at the macro-scale (>1 mm²). These assays detect and/or quantify the target analyte through detection and measurement of signal generated by large numbers of binding or lytic events. The signals generated by tens of thousands to hundreds of millions of discrete binding or lytic events are aggregated, typically through the interaction of all of the events with a single optical signal path providing a single result. One reason for this traditional approach is that the binding or lytic events to be detected occur on a molecular scale, and thus large numbers of events are required to create a detectable signal. Additionally, this large number of events creates a statistically meaningful basis for the result.

A clear limitation of this traditional approach is evidenced in the case of very low concentrations of analyte. The signal generated by sparse binding events must be great enough to be distinguished against the background noise. Alternatively, the signal generated must be differentiable against the field of negative signal caused by averaging the change in signal over the entire surface area of the reaction zone. In solid phase assays, the signal strength of this field is, thus, a function of the volume of sample or the area of the reactive surface. In these cases, the signal generated by sparse binding or lytic events incorporates the signal generated by the much larger unaffected region of the test area. In the case of very low concentration analytes this has the effect of creating a very small difference between a positive and a negative signal, in turn, limiting the lower level of detection that is achievable.

The instant invention is a solid phase detection method and system for biological markers where the frequency, density or distribution of the binding events is far below that which is detectable by traditional immunoassay, DNA probe, immuno-chromatographic or other ligand binding methods.

Immobilization

Solid phase methods are well known in the art of assay development as a means of separating, or capturing, an analyte of interest (“ligand” or “analyte”) from a multi-component fluid sample. Solid phase assays require a capture material (“receptor”) that is immobilized onto the solid phase that binds specifically to the analyte of interest, forming a ligand-receptor complex.

The ligand and receptor bind specifically to each other, generally through non-covalent means such as ionic and hydrophobic interactions, Vanderwaal's forces and hydrogen bonding. Certain ligand-receptor combinations are well known in the art and can include, for example, immunological interactions between an antibody or antibody Fab fragment and its antigen, hapten, or epitope; biochemical binding of proteins or small molecules to their corresponding receptors; complementary base pairing between strands of nucleic acids.

Solid phase immobilization of receptor material is well known in the are. General classes of immobilization include, for example, but are not limited to adsorption, covalent attachment, and linker-mediated. Adsorptive binding is generally non-specific and relies on the non-covalent-interactions between the solid phase and the capture material. Covalent binding refers to linking of the capture material to the solid phase via the formation of a chemical bond. Linker mediated immobilization involves the specific use of secondary molecules and/or macromolecules attached to the surface and capture material that interact specifically to form a bound structure. Immobilization methods are generally chosen so that the capture material retains its specificity for binding to the analyte of interest.

Once the capture material is immobilized to the solid phase, the solid support is reactive to analyte binding (“reactive surface”). Before the addition of a fluid sample containing the analyte of interest, it may be necessary to treat the reactive surface with additional materials to prevent (“block”) the non-specific binding (“NSB”) of non-analyte components of the fluid sample to be tested. Typical blocking materials include, for example, proteins such as casein and bovine serum albumin, detergents, and long-chain polymers.

Typically, the chosen receptor is immobilized to a solid phase. A test solution containing the analyte of interest comes in contact with the immobilized receptor whereby a ligand-receptor complex is formed on the solid phase. Once this complex is formed, all other components of the test solution are removed, usually by rinsing the solid phase. The analyte bound to the solid phase may be additionally complexed with a mass amplifying agent through a secondary specific receptor binding to form an analyte complex. This complex may be formed either in the fluid sample containing the analyte before the sample contacts the reactive surface, or after the analyte is bound to the reactive surface. After binding of the analyte or analyte complex to the reactive surface is complete, this binding can be measured by any of several means.

Substrates useful for creating the disclosed solid phase binding platform include all non-transmissive and transmissive materials suitable for optical or “near optical” wavelength reading. Suitable substrates include, for example, those substrates that provide sufficiently consistent or precise interactions with light in order to yield consistent and meaningful results. To that end, the use of highly absorptive surfaces or attachment layers may create optical contrast in the scattering applications disclosed herein.

Optical Signal Format

The Optical Signal Format of the instant invention is comprised of at least a signal carrier, a signal generator and a signal detector.

Optical Signal Format: Signal Generator

The present invention specifically relates to a method for altering the ratio of signal to non-signal surface area, allowing for more sensitive results. Also, this invention uses specific labels selected to interact with specific optical beam types to create an enhanced, differentiable or amplified signal.

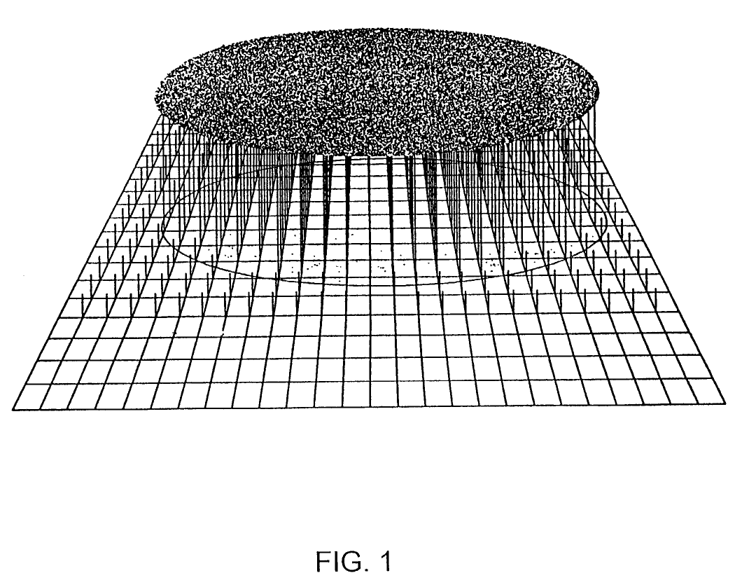

The traditional goal of a binding assay method is the determination of mass per unit volume (e.g., ng/ml) or equivalent (e.g., IU). See FIG. 1. A solid phase is typically used as a separation platform to isolate an analyte from other elements of a sample and from excess reagents. For certain types of assays, the signal generator remains attached to the binding complex, and thus is read from the solid phase (e.g., optical methods as discussed infra or fluorescence). The mass of analyte found in the volumetric sample is converted to mass immobilized on the solid phase in a proportional manner.

The signal generator, as used herein, is that component of the invention that interacts with a signal carrier to create a signal. Key to this concept is the known, specific and predictable interaction between the two. A signal generator element includes material which may be used to specifically label, amplify, distinguish, mark or generate a detectable signal associated with the immobilized target analyte, thus differentiating binding from the absence thereof.

Limitations on selection of a signal generator are driven by the selection of signal carrier, secondary reagent conjugation specificity, target analyte, and physical, chemical and/or electrical reactions. Within these limitations, a plethora of signal generators exists. These include, for example, material adding significant mass to the analyte complex, self-assembling, aggregating, enzymatic or chemically active materials, film-forming materials, materials generating optical signatures or distinctive optical properties, i.e., high refractive index, chiral properties, high absorption, high levels of scatter. Furthermore, multiple signal generators may be employed to create discrete signals for different binding events.

Light Scattering Labels

The signal generator component of the scattering embodiments disclosed herein may be referred to as a light-scattering label. A light scattering label is a molecule or a material, often a particle, which causes incident light to be scattered elastically, i.e. substantially without absorbing the light energy. Exemplary labels include metal, metal coated and non-metal labels such as magnetic particles, silica, colloidal gold or selenium; metal coated polymer or silica particles; and polymer particles made of latex, polystyrene, polymethylacrylate, polycarbonate or similar materials. The size of such particulate labels ranges from 5 nm to 10 μm, typically from 5 nm-5 microns, and preferably 5 nm to 900 nm. Suitable particle labels are available from Bangs Laboratories, Inc and Fishers.

In the present invention, the label is attached to either a secondary receptor (“labeled secondary receptor”) that binds specifically to the analyte of interest, or to an analog of the analyte (“labeled analog”), depending on the format of the assay. For a competitive assay format, the labeled analog specifically binds with the reactive surface in competition with the analyte of interest. For a direct sandwich assay format, the labeled secondary receptor is specific for a second epitope on the analyte. This permits the analyte to be “sandwiched” between the immobilized receptor and the labeled secondary receptor. In an indirect sandwich assay format, the secondary receptor is also specific for a second epitope on the analyte and is labeled with a material that specifically binds an additional light scattering label. For example, once an analyte is captured by the reactive surface, a biotinylated antibody may be used to sandwich the analyte, and an avidinated light scattering label is used for signal generation.

Regardless of the assay format, the receptor or analog must be attached to the light scattering label to form a “labeled conjugate.” As with the immobilization of the capture ligands to the solid phase, the light scattering labels may be covalently bonded to the receptor or analog, but this is not essential. Physical adsorption is also suitable. In such case, the attachment to form the labeled conjugate needs only to be strong enough to withstand forces in certain subsequent assay steps, such as washing or drying.

In the preferred embodiment, signal generators are conjugated to binding reagents, which in turn, allow specific interaction with the target analyte, analyte complex or immobilized capture material. Such signal generators include, for example, beads and microparticles and colloidal metals, as discussed previously. Signal generators may also include self-assembling and synthetic polymers, glass, silica, silial compounds, silanes, liquid crystals or other optically, active materials, macromolecules, nucleic acids, catalyzed, auto-catalyzed or initiated aggregates, and endogenous or exogenous sample components. Useful binding reagents generally include antibodies, antigens, specific binding proteins, carbohydrates, fectins, lipids, enzymes, macromolecules, nucleic acids and other specific binding molecules.

Optical Signal Format: Signal Carrier

Signal carriers useful in the instant invention are optical and near-optical pathways. These pathways interact with a signal generator such that single event detection is possible. Either monochromatic or multiple wavelength electromagnetic radiation reflected from or transmitted through the sample may be used to detect a change in signal.

Optical Signal Format: Signal Detection

Historically, the effect of the use of a single optical beam for reading the surface, e.g., a laser beam, is the production of a single result representing the mass change effects of all binding events within the assay area. Where a large beam is presented to the immobilized mass and the result is integrated by a single detector, the effective result is the same.

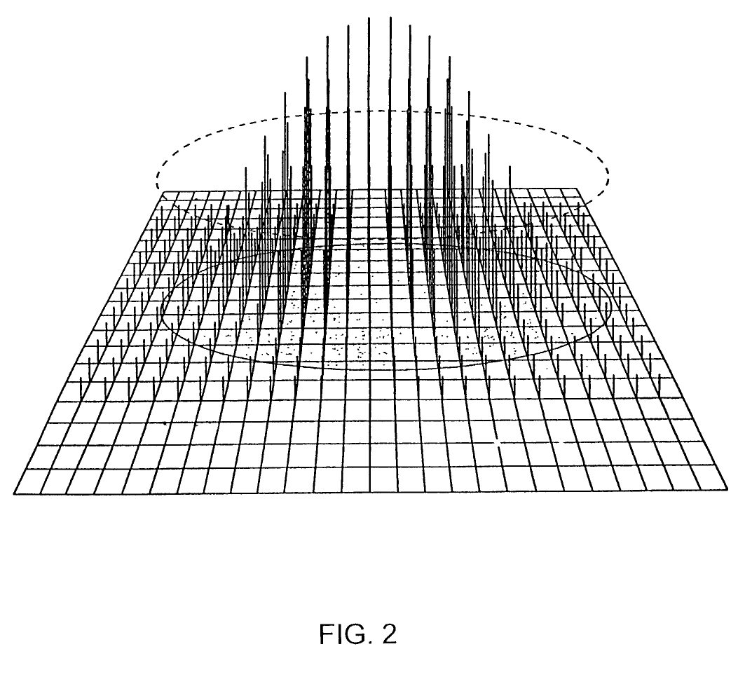

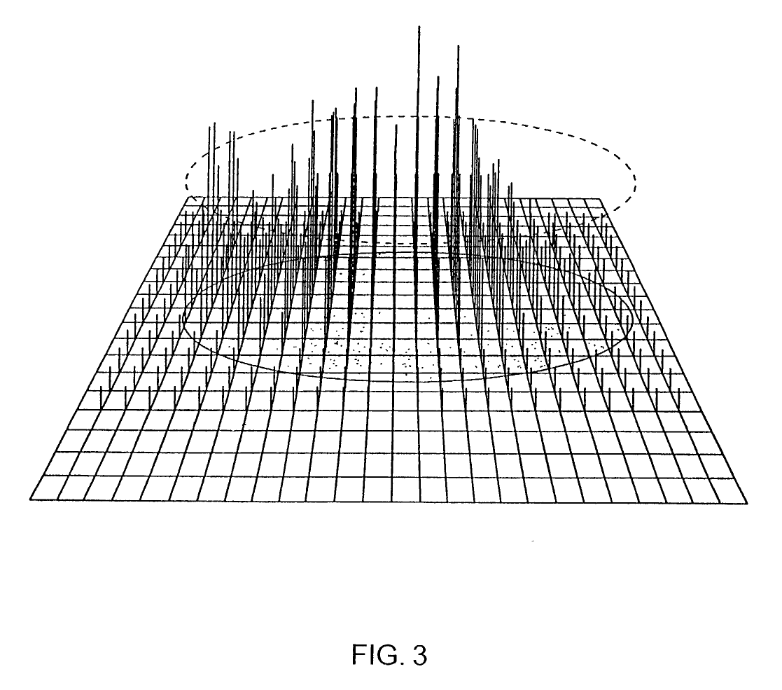

As shown in FIG. 2, the historically idealized model for this method is the optical averaging occurring over a statistically significant or an entire assay area; represented by an approximately normal distribution of binding events over the assay area. In virtually all actual cases, the binding distribution over the assay area is highly non-homogeneous. See FIG. 3. An advantage of the current optical ellipsometric read method employing a single large beam and single detector, hereinafter referred to as OTER™ (DDx, Inc.), is that it inherently integrates all of the binding events within the assay area without regard to distribution, aggregating countless individual binding events into a single average result.

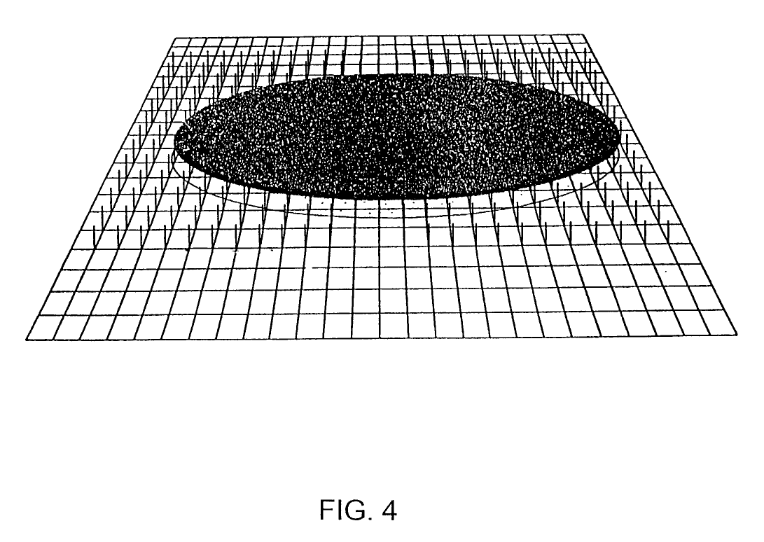

A disadvantage of this method derives from that same optical averaging effect. As depicted in FIG. 4, in those cases in which the target analyte is comprised of small molecular size particles or in which there are sparse binding events, this method tends to cause results to be statistically reduced to insignificance when averaged over this relatively large assay area. Consequently, results that involve very low concentration positives are indistinguishable from negative results against background noise or variability of the assay system.

One embodiment of the instant invention involves a novel microbiological use of ellipsometric methodologies, that is, the determination of individual binding events via enumeration. This method solves the signal averaging problem by dividing the surface being analyzed into a large number of discrete “local” detection areas. Any signal generated within such a local reading zone is averaged over a much smaller area or field, and thus is “diluted” against an otherwise negative background to a much smaller extent.

For low concentration analytes this method generates numerous local results for any given test surface, most of which report negative results. However, in those cases where positive binding has occurred, the local reaction zone reports a very high positive signal; the averaging over the entire area has not diluted the positive signal. Thus, a non-integrated result profile is generated thereby reporting discrete positive results over a total test area that may be by in large negative, while allowing for much larger individual signals to be generated for local positive events.

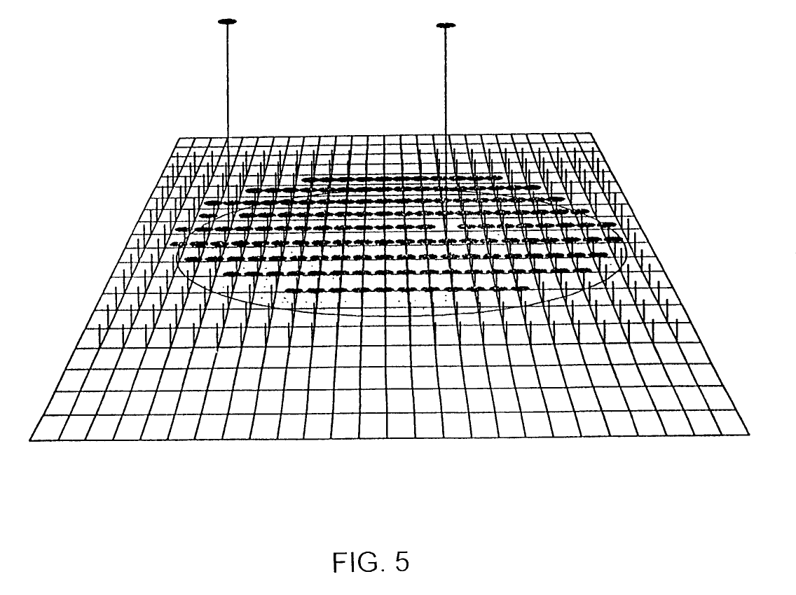

The enumeration methodology, thus, allows for extremely sensitive assay procedures, including the determination of individual binding events. An obvious application of this method (as referenced in FIG. 5) is in microbiology for the detection of low numbers of microorganisms. The ability to detect individual cells or clusters of cells (colony forming units) enables the elimination of time consuming culture steps. This is particularly important for those pathological organisms for which the presence of even a single organism must be considered a positive result. That is, a zero-tolerance level. Another useful application of the instant invention is in hybridization assays, wherein the reaction product exists in extremely small quantities. In this case, individual binding event detection eliminates the need for cumbersome amplification techniques, for example, PCR, NASBA and SDA. All assay systems having clinically relevant thresholds of detection below those readily achieved by traditional assay methods benefit from this invention.

The enumeration principle is illustrated in FIG. 5 using a small beam diameter, to provide a local reading area. This beam provides a vastly higher relative signal for discrete binding events, as averaged over a much smaller spot area. More specifically, a collimated beam of light is scanned over a test piece in a raster (X-Y) fashion. The beam, outside diameter (OD) approximately 20 microns, scans over a cell or group of cells evidencing drastic changes in the reflected light properties as received at the detector. The amplitude of those changes depends on, for example, the size of the optical beam and/or the size of the cell or cell groups. In particular, a cell that is small in comparison to the beam will be difficult to detect above general noise associated with background light and detector amplification. The closer the beam OD and cell size approach each other, the larger the optical property changes. Practical light sources for application of the instant invention include a beam having an OD ranging approximately from 5-50 microns, i.e., laser diodes. Laser diodes are compact in size and utilize small diameter lenses to manipulate light, thus, facilitating variable equipment dimensions, for example, bench top, lap top and hand held equipment. Moreover, a CCD detector could result in a significant improvement in sensitivity and shorten assay run time. A fundamental difference between the OTER and enumeration approaches, thus, is the optical. pathway employed.

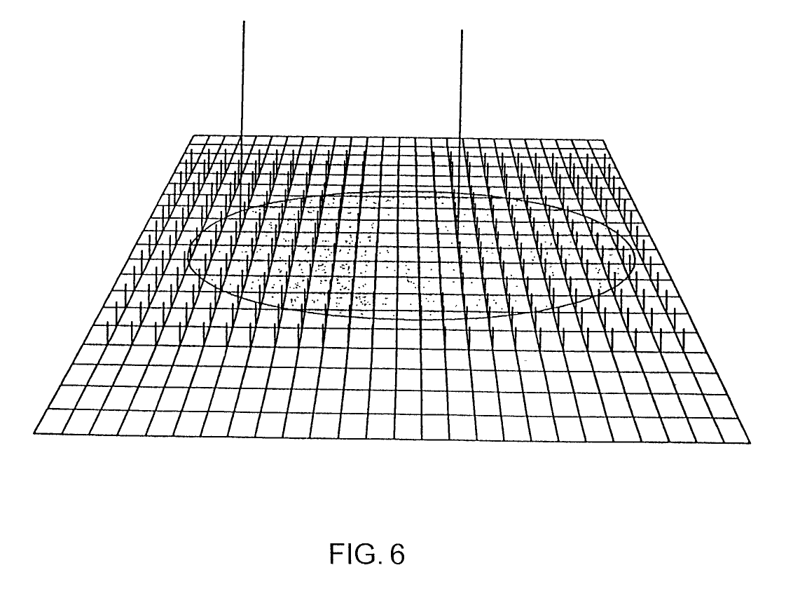

A signal detector, in general, must be receptive at the wavelength of the signal carrier and must be configured to receive the system information. Signal detectors may include CCD cameras, single silicon detectors and diode array detectors. An ellipsometer in conjunction with CCD looks at the entire reaction zone and breaks it up into areas. Thus, there is a need to eliminate the negative areas and sum the positive areas. The invention disclosed herein magnifies a spot on the reaction zone and breaks that spot into areas, looking for individual binding events, e.g., beads, cells, colony forming units. FIG. 6 depicts topological resolution of the surface evidencing enumeration of individual binding events.

It is, in fact, because the binding events are not integrated over the surface that this method is used to approximate individual or discrete binding event identification. Key to practicing the enumeration method, is the ability to segment, parse or segregate discrete areas of signal for highly focused readings, thereby, increasing the ability to discriminate a positive from a negative result. Signal parsing may take place either within the carrier aspect or the detector aspect of the invention. These results are displayed as a series of discrete signal values and compared to a predetermined cut-off point, thereby determining positive binding events within any local read zone. In this manner individual binding events are enumerated on the surface, with a resolution determined by the size of the read zone. To change the relative aspect ratios of the true signal versus background signal or noise involves changing the amount of background over which any true signal is averaged. A constant signal, averaged over a progressively smaller background signal becomes progressively more distinct, until individual signal generators are readily enumerated.

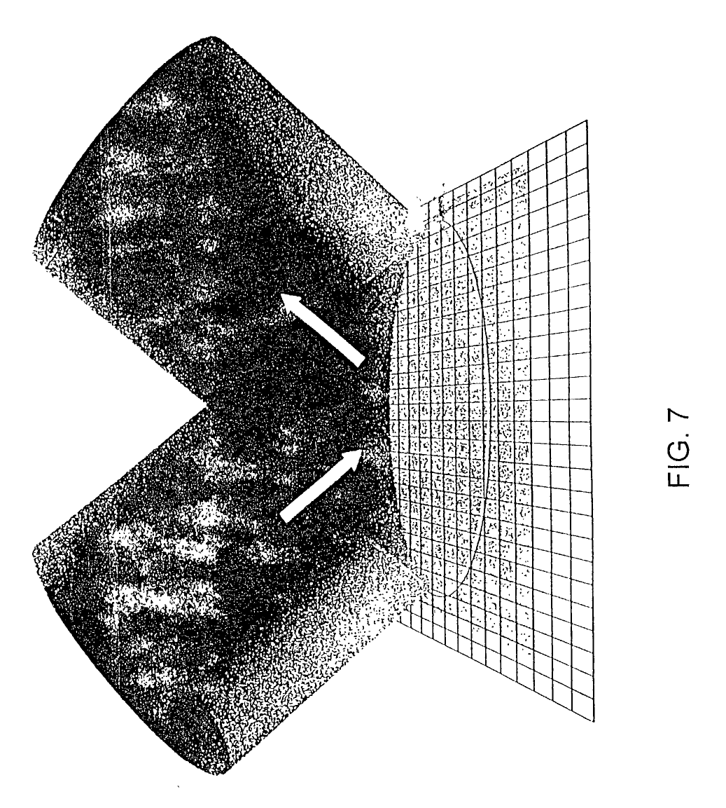

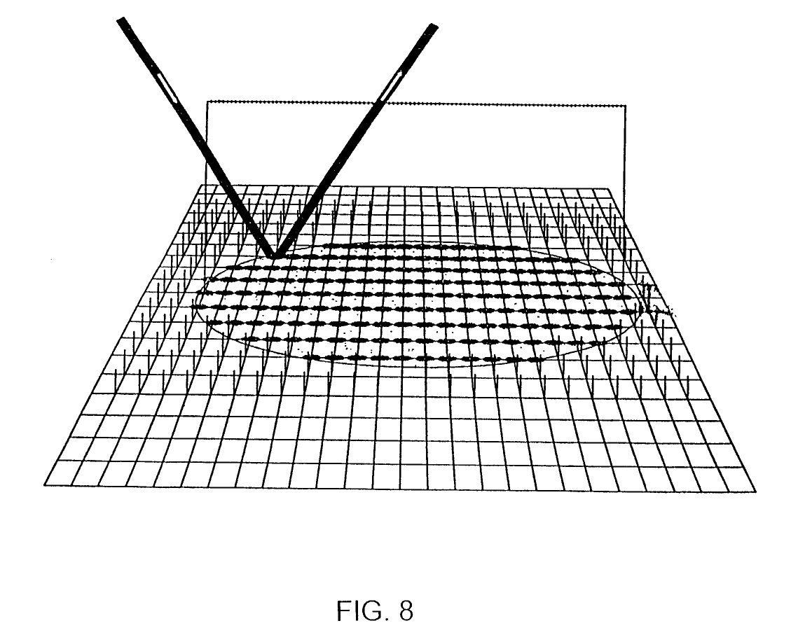

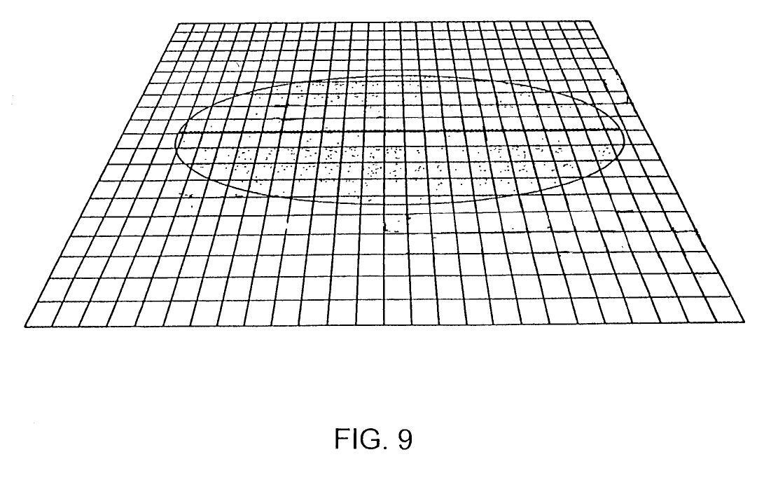

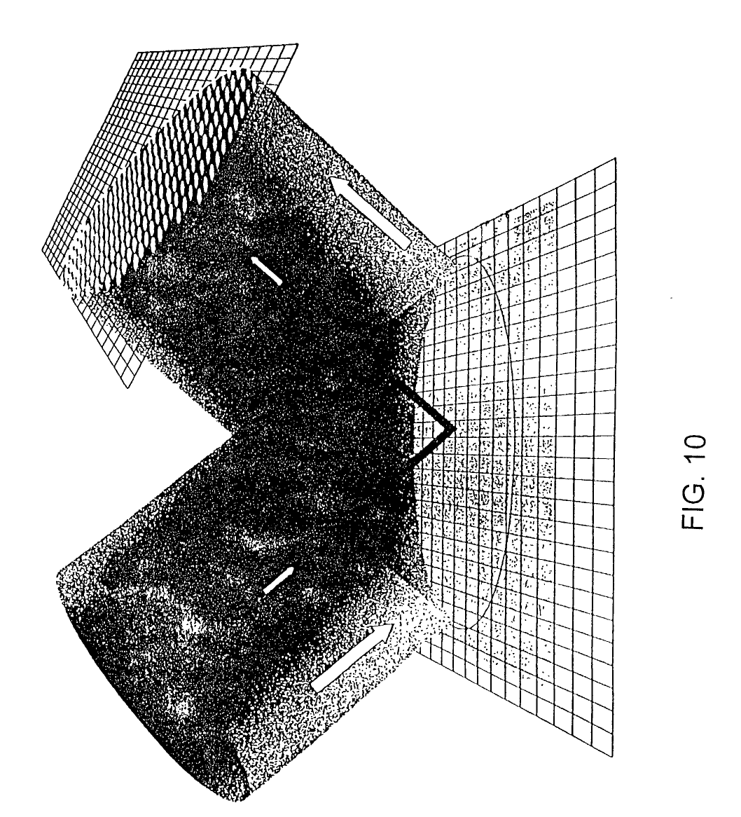

FIGS. 7 and 8 compare the differences between the current OTER instrument configuration and one of the enumeration capable instrument configurations. The intersecting beam in the OTER configuration has a surface area of approximately 13 square millimeters (Pi*r²=SA (mm²)=3.14159×2²=12.6566 mm²) over which any positive binding events are averaged. Signal parsing by the use of a much smaller diameter beam is illustrated in FIG. 8 (i.e., 20 μm). The beam is scanned across the surface, taking discrete local readings over the same total surface area. In this example, the reaction zone is 2 mm in diameter, and the scanning beam is 20 μm in diameter. Using standard conversions (see FIG. 9), the total reaction zone surface area is 3,141,590 μm², while the small scanning beam reads 314.159 μm² at each local zone. With 100 discrete measurements along the diameter, a 20 μm beam makes 10,000 discrete readings withing the reaction zone.

An inherent signal is generated by each binding event. That signal is not altered by the reduction of the reading zone. Each event generates the same response locally as it would in the OTER configuration. However, the area over which this signal is averaged is reduced 10,000 times, thus, effectively amplifying the signal against the background by 10,000 times in the enumeration system. This change represents an enormous increase in the ability to differentiate a positive result from a negative result, effectively improving the lower limit of detection (chemical sensitivity or threshold of detection) of the assay method by 10,000 times.

FIG. 9 represents preliminary calculations as to the limits of detection possible using the OTER and the enumeration approaches. The specific number and examples chosen are not significant to the disclosure, and should not be interpreted as limiting its scope. Rather, they are included herein as an example of the sensitivity differences possible between the two systems. Enumeration is able to detect a single binding event, and as few as 100 binding events generate a clearly enumerable positive result over the system and biological noise. The probable limit of detection for an unamplified OTER system under comparable circumstance is 2×10⁶ cfu/ml. The addition of mass to the system via amplification does not result in substantial improvement of sensitivity due to the pervasive effect of area averaging.

Signal parsing may also take place at the detector. Through the detector system, an aggregate signal may be divided into discrete information pathways correlating to discrete areas on the test-piece using a broad or large beam width. For example, a CCD or diode array detector may be used in this manner. In cases such as this, the parsed signals must be kept discrete and proportional through the detection and reporting process; magnification, focus and carrier detector position control are methods for keeping information commensurate throughout the system. The use of a monolithic or single crystal diode detector requires the signal to be divided into suitable small units within the signal carrier.

An alternative embodiment to the small beam scanning approach is the use of a CCD or diode array to read and parse the laser beam into smaller discrete signals. The object of this embodiment remains the determination of small spot response within the large beam spot area. However, in this case the definition of the small read zone (local result) is not provided by the diameter of the intersecting beams, but by the arrangement of the detector receiving the beam. Further, the detector, such as a photo diode array, CCD or other non-integrating signal receiver, receives the information contained in the large beam, and preserves this information as smaller local results for processing. This effectively creates a large number of virtual beams, defined by the path that the light intersecting the array as a specific detection point has taken, all operating simultaneously. The aggregate signal for all virtual beams equals the large beam signal—each virtual beam references only a limited surface area—and the results are not integrated together.

An advantage of this method is that it is rapid (parallel signal processing). The scanning approach is a serial process in which each reading is made in sequence. Additionally, the technical challenges of producing this embodiment are substantially less than those involved in the development of a small beam laser and an accurate scanning control mechanism.

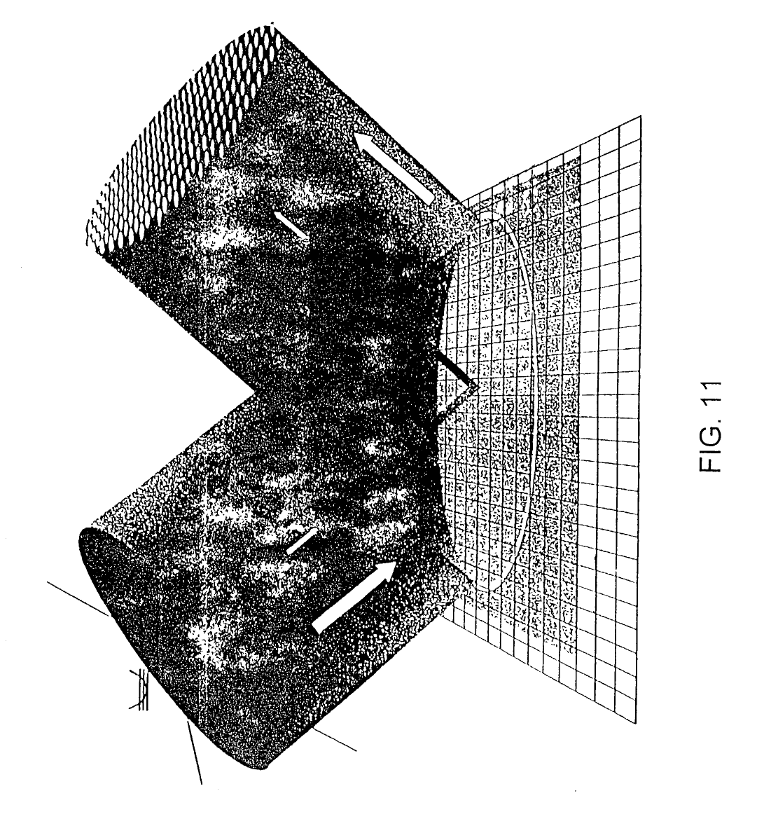

As discussed supra, a variety of optical signals may be used within this system. The specific optical signal is selected to provide the appropriate level of information, based upon the nature of the material to be detected, and the resolution desired. The examples provided herein use ellipsometry and scatterometry, see FIG. 11. However, a variety of optical methods will be substantially improved by adopting the general concepts and methodologies described herein. In particular, effects such as absorption, refractive index change, chiral effects and diffraction may be used within essentially similar optical configurations. FIG. 12 lists possible optical signal types, thus, displaying the range of methods amenable to the enumeration approach. It is neither limiting nor intended to comprise a complete listing thereof.

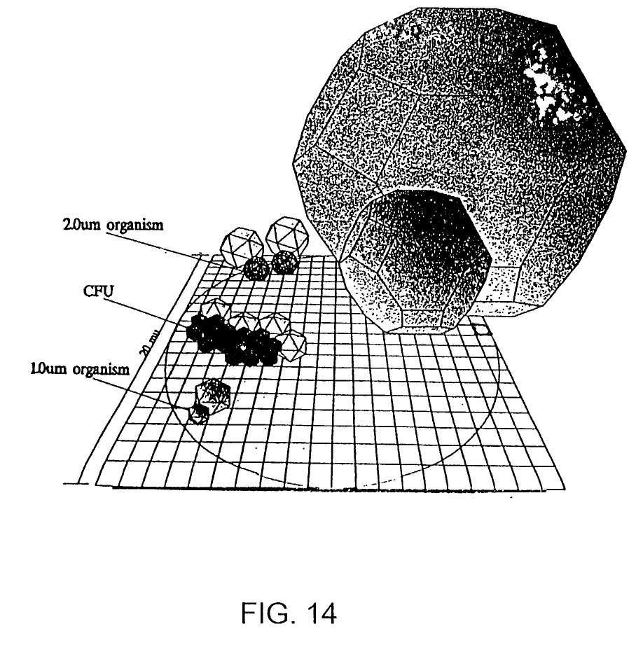

Mass enhancement labels can play a central role in the practice of the enumeration method at high sensitivities. FIGS. 13 and 14 illustrate, proportionally, the aspect ratio or relative height:width:breadth of various size materials that may be used as signal generators. As is diagramed in these figures, organisms at the cellular scale generate very significant signal without amplification within the system. In comparison, the thin attachment layer represented along the bottom of the reading zone surface creates a clearly distinguishable signal with the current OTER format. The signals generated by mass contained in the much larger objects used as labels significantly improve sensitivity.

Additionally, for either the scanning (small beam) or the array (virtual beam) approach as discussed, a substantial improvement in signal detectability is possible using unique characteristics of optically based mass detection systems. Particular properties of any given mass enhancement label may be used to alter the optical signal based upon its physical characteristics, including its effect on optical characteristics: refractive index, scatter, chiral effect, general adsorption, wavelength specific adsorption and diffraction.

Use of selected labels to induce unique or distinct optical effects creates an improved ability to discriminate the signal generated by the binding of label to the complex from that created by surface background or in the absence of specific binding events. This operates through the creation of an enhanced or attenuated apparent signal over that which would be created by normal materials.

FIG. 14 specifically provides an example of this type of effect through the use of high refractive index material in an ellipsometric format. Because the change in polarization state detected by ellipsometry is caused by two distinct factors (absolute mass and refractive index) the use of a high refractive index material as the mass enhancement label effectively increases the apparent mass detected by the ellipsometer, thus, further amplifying the signal from the binding event.

Any number of optical interactions with specific types of material designed to amplify or enhance the strength of the signal, or to create a unique signal type, are envisioned and are included herein by reference.

Detection of scattered light (scatterometry) may occur visually or by photoelectric means. For visual detection the eye and brain of an observer perform the image processing steps that result in the determination of scattering or not at a particular situs. The terms “situs” and “site” refer, herein, to the area covered by one ligand. Scattering is observed when the situs appears brighter than the surrounding background. If the number of sites are small, perhaps a dozen or less, the processing steps can be effected essentially simultaneously. If the number of sites is large (a few hundred or more) a photoelectric detection system is desired.

Photoelectric detection systems include any system that uses an electrical signal which is modulated by the light intensity at the situs. For example, photodiodes, charge coupled devices, photo transistors, photoresistors and photomultipliers are suitable photoelectric detection devices. Preferably, detector arrays (pixels) correspond to the array of sites on the reactive surface for signal parsing, some detectors corresponding to non-situs portions. More preferred, however, are digital representations of the reactive surface such as those rendered by a charge coupled device (CCD) camera in combination with available frame grabbing and image processing software. The image processing techniques preferred in the instant invention can be derived from “IMAQ for Vision Tool Kit” available from National Instruments Corporation of Austin, Texas and which is compatible with the Labview programming environment.

A CCD camera or video camera forms an image of the entire reactive surface, including all label and non-label areas, and feeds this image to a frame grabber card of a computer. The image is converted by the frame grabber to digital information by assigning a numerical value to each pixel. The digital system may be binary (e.g. bright=1 and dark=0) but an 8-bit gray scale is preferred, wherein a numerical value is assigned to each pixel such that a zero (0) represents a black image, and two hundred and fifty-five (255) represents a white image, the intermediate values representing various shades of gray at each pixel.

Data Analysis

The digital information may be displayed on a monitor, or stored in RAM or any storage device for further manipulation, such as imaging printing and archiving. Image processing software, such as “IMAQ for Vision Tool Kit”, is used to analyze the digital information and determine the boundaries or contours of each situs, and the value of intensity at each situs. “IMAQ for Vision Tool Kit” is commercially available software for digital image acquisition, processing and analysis. “IMAQ for Vision Tool Kit” automatically counts and measures objects within an image, after which it sorts and classified the objects by specific characteristics, including, for example: angles, area, length, width, diameter radius perimeter, area or aspect ratios, color, position, optical density and hole areas. “IMAQ for Vision Tool Kit” is also able estimate the number of objects contained within a cluster of objects.

“IMAQ for Vision Tool Kit” may be programmed to perform a specific series of functions and analyses in order to differentiate true analyte complex particles form other particles or optical features, e.g., dust, non-specific binding, solid phase anomalies, masking. That is to say, the object measurement characteristics discussed herein may be used to create signal:non-signal filters.

Often, the image will require enhancement to improve the software's ability to enumerate individual binding events. Enhancement techniques may include, for example, brightness:contrast adjustment and spatial:morphological filtering. More specifically, there are three basic categories of image enhancement: intensity index modification, spatial filtering and image frequency manipulation.

Modification of the intensity index is directed to a change in the way intensity values of each pixel are interpreted. Aspects of the intensity index include, for example, brightness, contrast, gamma correction, thresholding, background flattening, background subtraction and intensity equalization.

Spatial filtering techniques analyze and process an image in small regions of pixels. Specifically, by reducing or increasing the rate of change that occurs in the intensity transitions within an image. This filtering includes convolution (linear) and non-convolution (non-linear).

Manipulation of the image frequencies is directed to the elimination of periodic or coherent noise in an image by converting the image to a set of frequencies, and editing out the frequencies causing the noise problem. A common technique used for this is the Fourier Transform.

It is envisioned that the digital image processing functions necessary may be consolidated into a laboratory-based instrument adapted for and capable of semi- and/or automatically performing all software-based steps of enumeration. It is not an essential element of the invention to display the surface image. It is essential only that the software image processing is performed entirely with the data provided by the digitization of the image.

The inventive clustering process as described in U.S. Pat. No. 5,329,461 may be adapted for utilization in a variety of applications to spatially resolve and count discrete analyte particles or individual binding events in conjunction with the instant invention. For example, detection of analyte particles comprising a molecule and a label for rapid scanning to locate areas of interest within an image of a sample.

Instrumentation

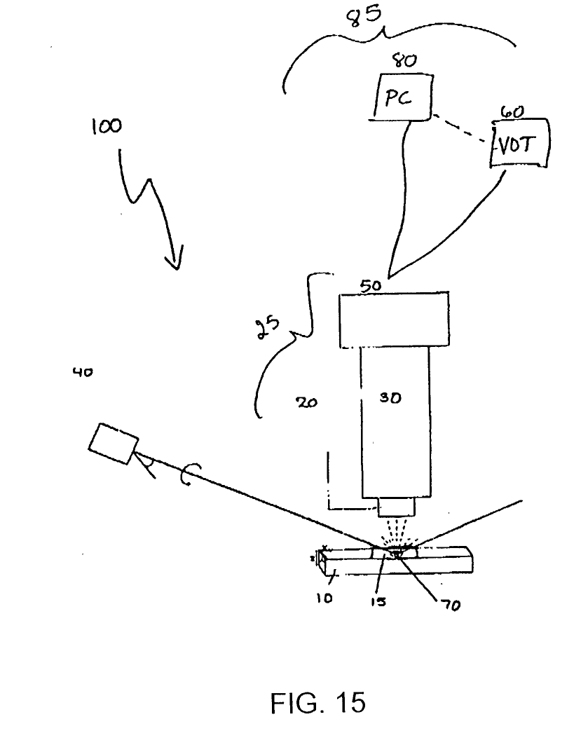

With respect to analyzing a test piece, an embodiment of an instrument for obtaining data and making determinations using light scattering principles is illustrated in FIG. 15. Generally, and referring to FIG. 15, a prepared test piece is secured to the sample stage and manually positioned such that the center of a test spot is aligned with the center of the objective lens. The test piece may be prepared to contain multiple test spots, therefore, to begin the test spot designated as 1, or first, is centered. Using the sample stage's translational capabilities (the detector could be alternatively or additionally moved, manually and/or automatically), the detector is manually focused on the scattering particles. Next, the image produced by the light scattering is collected and saved. Finally, the sample stage is translated to two alternate locations, one each to the left and right of center, and image acquisition repeated at each location. Each generally herein-described step in the detection process may be repeated for any number of test spots contained on a test piece.

The instrument employed for the enumeration methodology disclosed herein consists of three defining modules: a sample stage, an optical signal format corresponding to the immobilized analyte complex, and a means for data collection and analysis. Each module is adapted for independent translation on at least two axes, thereby facilitating optimal optical effect, alignment and focus. The instrument and its modules, in toto, are fixed and stationary in relation to one another by standard attachment means to, for example, a solid, planar, horizontal platform. More specifically, as shown in FIG. 15, the enumerator 100 is comprised of a means for data collection and analysis 85 consisting essentially of a computer 80 and video display terminal 60 functionally combined with a sample stage 10 and optical signal format consisting essentially of a signal carrier 40 and a signal detector 25 configured such that when a signal generator, such as a light scattering label, is irradiated, it is able to be detected by the enumerator 100.

The sample stage 10 may be any planar stage or platform adapted for receiving and securing thereon a mounting jig 15 onto which a test piece 70 is secured to the mounting jig 15. The test piece 70 may be secured by any suitable means, such as, double sided adhesive tape or a mechanical mounting means. The stage 10 translates on at least an X-Y axis basis, and in the preferred embodiment, also possesses additional rotational and angle control. The test piece 70 is further comprised of test spots, prepared as described herein.

The optical signal format is comprised of a signal generator such as a light scattering label bound to a test spot as described herein, a signal carrier 40 and a signal detector 25. In the preferred embodiment the signal carrier 40 is an electromagnetic radiation source, and more preferably, a laser diode adoptively mounted to possess both rotational and angular control. The signal detector 25, an integrally combined microscope focus tube 30 and objective 20 functionally combined with a photodetector, and preferably a CCD camera 50 are movably disposed, by any standard movable mounting means, vertically above the sample stage 10. One or both of the objective 20 and the signal carrier 40 (e.g., laser diode) are movable so that the output laser beam is focused at the center of the objective 20 lens focus. The signal detector 25 is functionally combined by standard means with the data collection and analysis means 85 comprised of a PC 80 and video display terminal 60, each of which is accordingly appointed with appropriate software and electronics.

In use, the PC 80 and video display terminal 60, and signal carrier 40 are powered on and allowed to warm up for at least 30 minutes. While the unit is warming up, the test piece 70 is adhered to the mounting jig 15, which in turn, is secured to the sample stage 10 directly and vertically below the signal detector 25. The test spot on the test piece 70 that has the target analyte bound thereto is then centered, aligned and focused between the signal detector 25 and the signal carrier 40. The enumerator 100 is engaged, an image acquired and exhibited and/or stored accordingly. The test piece 70 is realigned for additional image capture to the left and right of the test spot, as described herein. Engagement of the enumerator 100 and image capture is repeated in a similar manner for each of the test spots on the test piece 70.

Prior to engagement of the enumerator 100, the appropriate software preparation is performed. For example, subfolders, default settings and macros are setup. Generally, light scattered by surface-bound microspheres is collected and magnified by a microscope objective lens and focused onto a CCD array, e.g. 640×480 pixels. CCD signal output is fed to both a black and white monitor and a data translation frame grabber such as Data Translation DT3155 high accuracy scientific frame grabber (Data Translation, Inc.). Image acquisition and analysis of the image formed by scattered light is accomplished with software adapted for and/or specifically directed to such function, for example, “IMAQ for Vision Tool Kit”.

Data analysis that includes discrimination and counting of scattering objects within an image is performed by software designed for such a purpose. Customized functions adapted into such software via, for example, macro programs, include exclusion of non-binding events from the object count by filtering, image intensity averaging and binary filtering. An example of a macro adapted for use in the preferred embodiment of the invention includes: transformation of bright scattering objects into a standard 3×3 cross; application of a watershed filter to the resulting cresses to separate scattered objects; determination of mean image intensity and the standard deviation of that mean; determination of a lower limit intensity threshold for a binary filter based on the mean image intensity; application of binary filter with threshold values of lower limit; and, automatic count of resulting objects having a mean diameter, for example, less than 10 pixels. The number of objects counted for each image is averaged over the three images produced for each test spot—center, left and right.

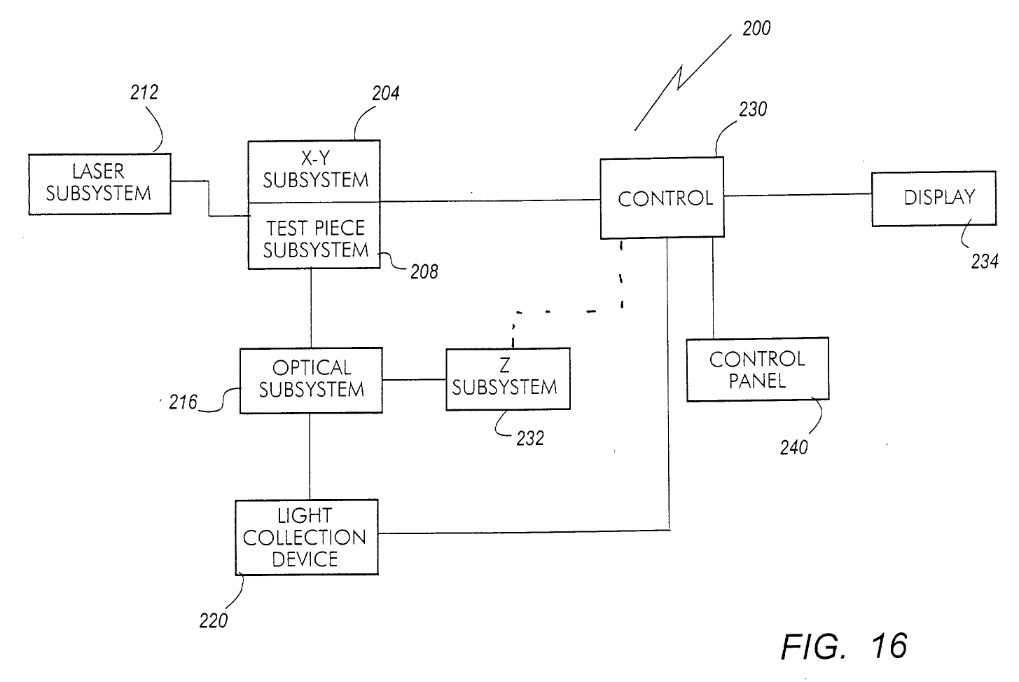

With reference to FIG. 16, a block diagram of a particular instrument 200 for determining whether a substance of interest, such as a particular or target analyte, is present with a sample under test is illustrated. The sample under test, in this embodiment, is movable in controlled X and Y directions using a X-Y subsystem 204. The test piece subsystem 208 is held to the X-Y subsystem 204 and moves therewith. The test piece subsystem 208 preferably includes a test piece having a number of test spots that contain one or more samples that are to be tested for one or more substances of interest. In a preferred embodiment, each of the test spots has a number of test subspots. Each of the test subspots may have only one substance of interest, although one or more of the subspots may have a different substance of interest, which, in one embodiment, is to be detected (if present) and not detected (if not present). In another embodiment in which there is an indirect assay format, a detection is made when the substance of interest is not present and a detection is not made when the substance of interest is present.

The test piece subsystem 208, in one embodiment has a silicon substrate and there are 12 test spots of about 6 mm in diameter. Each test spot is separated, in this embodiment, by 7 mm on center from each adjacent test spot. However, these test spots can be of different diameter and the distance therebetween is programmable or variable and can depend upon the sizes of the test spots. The X-Y subsystem 204 supports the test piece subsystem 208 in a manner that preserves the flatness of the silicon substrate so that, when in focus, it is in focus along all positions on the entire test piece of the test piece subsystem 208. In that regard, this support of the X-Y subsystem 204 is machined to be flatter than the silicon substrate of the test piece. Wire clips retain the test piece in position. External forces applied to the test piece can affect the flatness thereof, sometimes requiring additional focus steps along the length of the entire test piece.

With respect to obtaining data that is to be used in determining whether a substance of interest is present on a certain test spot and/or test subspot, a laser subsystem 212 is provided that includes a laser device (e.g., laser diode) that outputs a laser or light beam. The laser device can include an electrical drive circuit and a low voltage unregulated DC input from a common wall transformer. The electrical drive circuit regulates the input voltage to produce constant light output independent of voltage input. In one embodiment, a relatively low power, 5 milliwatt laser is used having fixed collimations. In another embodiment, a greater output powered laser, 30 milliwatt, is used to increase the light levels for detecting objects or particles (representing a substance of interest) of smaller sizes. The 30 mW laser can produce a large rectangular focused area that is at least as large as the current image area of the test spot (field of view) under test. It is preferred that the laser focused area or spot be larger than the field of view in order to make sure that the entire test spot then being tested is subject to uniform illumination. The 30 mW laser also has an adjustable collimation feature that allows its focused area or spot size to be adjusted to match the intensity and size of the test piece spot(s).

The instrument 200 also includes an optical subsystem 216 that gathers the light scattered from the test spot and/or test subspot of the test piece subsystem 208 to which the light beam from the laser subsystem 212 was applied. Different embodiments can be employed characterized by their magnification (e.g., 2×, 4× and 10×). A standard microscope objective and tube lens can be utilized for the 10× magnification. Regarding the 2× and 4× magnifications, commercially available lens hardware can be selected, such as InfiniStix from Infinity-Photo optical. It is desirable to select lenses that minimize, or at least reduce, the need for movement of those parts of the optical subsystem in the Z direction. To achieve this objective, the depth of field for the lens hardware must be greater than the Z motion error along the entire travel of the test piece in the X direction over the full range of travel. Proper selection of such lens hardware for the 2× and 4× magnifications can eliminate the need for movement in the Z direction and thereby render unnecessary automated Z direction motion. Instead, a one time micrometer adjustment, when such lens hardware with these magnifications is used, is satisfactory. The optical subsystem 216 is vertically mounted and adjusted so that at the lowest mechanical position of the vertical or in the Z direction there is no contact with the test piece of the test piece subsystem 208. The embodiments with the 2× and 4× lens hardware allow a relatively larger range of laser beam angles to be utilized, particularly in comparison with the lens hardware that has the 10× magnification in which only relatively larger laser beam angles can be utilized due to the proximity of the objective to the test piece surface, typically about 2-5 mm.

The scattered light received from the test spot and/or test subspot by the optical subsystem 216 is focused and applied to the light collection device 220 of the instrument 200. The light collection device 220 can be a high resolution monochrome digital camera. When objects or particles, indicative of the substance of interest are present with the sample defined used the test spot and/or test subspot, and such light is received by the light collection device 220 through the optical subsystem 216, such light appears as bright spots on a dark background. In one embodiment, the digital camera is a Sony XCD-SX 900 FireWire camera having a high resolution of 1200×960 elements that can include or be defined as pixels. Each pixel is 7.5 μm² in size. The sensor in this camera is an interline progressive scan CCD (charge coupled device) sensor with rectangular pixels. This sensor is capable of variable frame rates and is externally triggerable. The pixels associated with the light collection device 220 can be mapped to one or more specific sized test spots and/or test subspots, depending upon the selected or particular magnification. The light collection device 220, which can be embodied in such a digital camera, has an integration time that can be controlled by the operator or user. Generally, the integration time is controlled to achieve the best, or at least a desired, contrast in images being obtained. A greater integration time associated with the light collection device 220 is desirable when the objects or particles associated with the substance of interest, if present, are relatively dimmer. Conversely, when the objects or particles of the substance of interest are relatively brighter, less integration time, as dictated by shutter speed, is needed. A further parameter that can be controlled by the operator or which can be automatically determined or selected is the gain of the digital camera. The gain relates to signal strength and is useful in controlling the strength of the signals produced as a function of the scattered light being collected. The digital camera of the light collection device 220 is able to supply a continuous stream of images in real time or obtain an individual image for desired processing or for storage for later processing. An analog camera could also be used. A frame grabber could be used to convert analog data to digital data. The resulting digital data can be in black and white or in color, as can the digital data when a digital camera is utilized.

The instrument 200 also includes a control 230 that can be comprised of a computer having one or more processors. The computer executes all software required to control the instrument 200 and outputs results including test results concerning any presence of the substance of interest. The control 230 regulates movement of the X-Y subsystem 204 and can control the operation of the light collection device 220, which is preferably the digital camera. The computer of the control 230 can communicate with the digital camera of the light collection device 220 and the X-Y subsystem 204 through FireWire bus cables. In one embodiment, the control 230 also includes a FireWire controller card that communicates with the computer, a motion control index or sequencer controller and motion control driver amplifier used in controlling movement of the X-Y subsystem 204. With respect to control of the X-Y subsystem 204, the control 230 can include a X-servomotor with encoder and a Y-servomotor with encoder, which are activated or energized to provide the desired and controlled X and Y movements, respectively. Each of these DC servomotors can be driven using amplifiers. Signals from the X and Y motor encoders directly interface to the control index or sequencer controller. In one embodiment, the X and Y movements have a resolution of 0.36 microns. In addition to such X and Y motion control, the position of the lens hardware of the optical subsystem 216 can be controlled in the Z direction using a Z subsystem 232. In one embodiment, such control is a form of a manual positioning thereof, with the amount or distance of such positioning depending on the magnification associated with the particular lens hardware, such as whether it is 2×, 4× or 10×. Once the particular lens hardware is properly positioned in the Z direction for proper focusing, no further movement or position thereof may be required. That is, the lens hardware can maintain that same Z position for testing of numerous spots and/or subspots for one or more test pieces of the test piece subsystem 208. In another embodiment, automatic focusing can be provided the optical subsystem 216 using the control 230. In such a case, like the X and Y motion control, there can be a Z axis servomotor and accompanying encoder. In one embodiment, available movement in the Z direction is greater and can be substantially greater, such as greater than four times more available movement in the Z direction than in each of the X and Y directions. On the other hand, finer resolution can be provided in the Z direction, for example, movement in the Z direction can be as small as 0.125 micron.

A monitor device or other display 234 communicates or is associated with the control 230. The display 234 can output visual displays or representations, such as those related to test information or test results. As will be discussed later, the display 234 can display a histogram related to light intensity of received light as a function of pixels that are part of the digital camera of the light collection device 220. Information from the histogram can be used in conducting analysis associated with determining whether a substance of interest is present, as will be subsequently explained. Also in communication with the control 230 is the control panel 240. The control panel 240 can function as an input unit to permit the user or operator to select parameter settings, perform operations and conduct analysis. More information related to the control panel 240 will also be provided later.

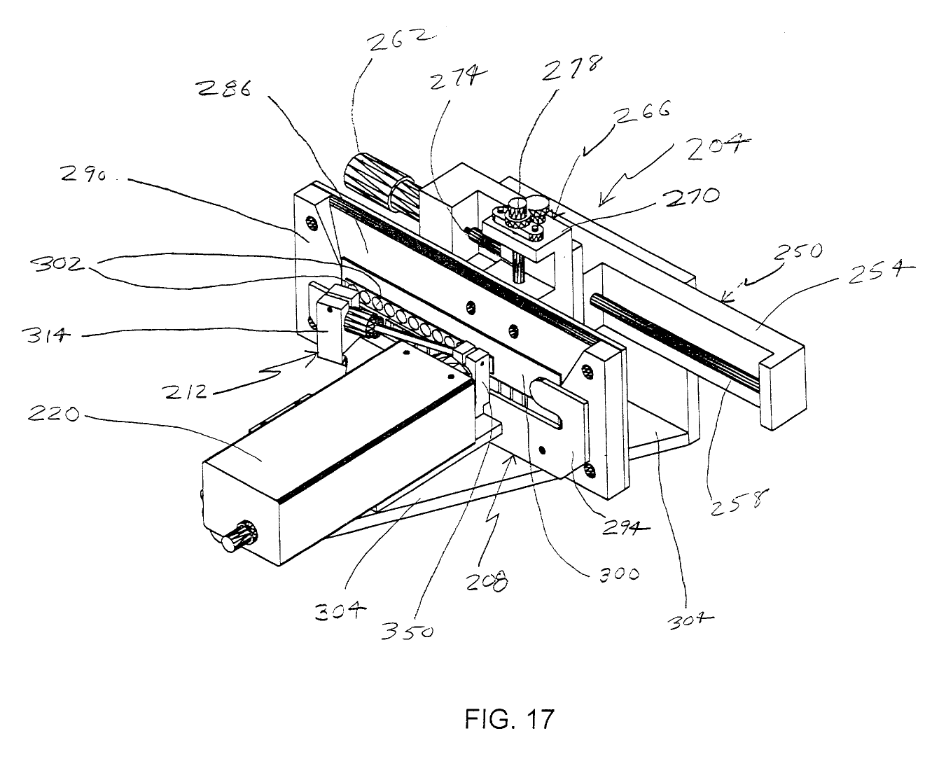

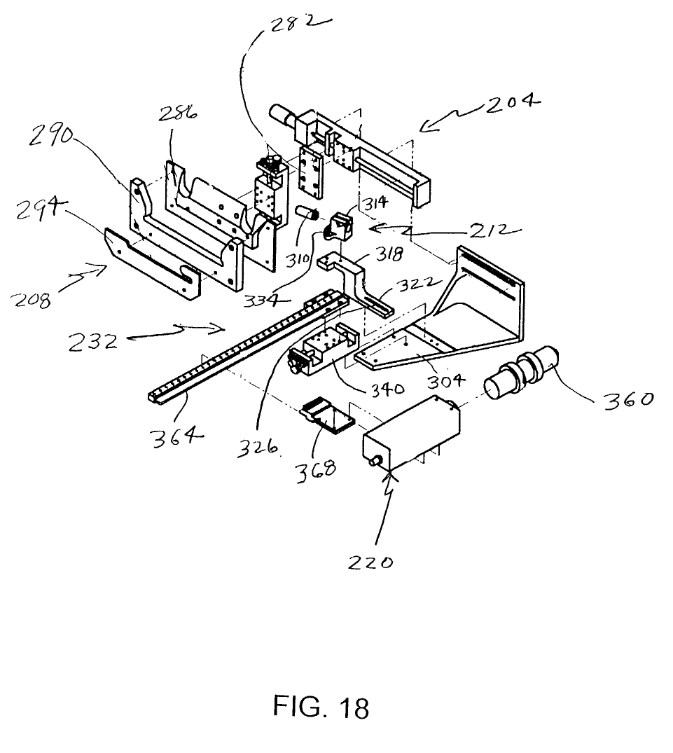

Referring to FIGS. 17-19, greater structural and operational details are described in conjunction with an embodiment of the X-Y subsystem 204, laser subsystem 212, optical subsystem 216 (FIG. 16) and light collection device 220. In this embodiment, the X-Y subsystem 204 includes a X subsystem 250 used in enabling movement in the X direction. The X subsystem 250 includes a frame 254 and a X-rod or track 258. The X-rod 258 is joined to a X-connector 262 that communicates with the output of the X servomotor. The rotational output of the X servomotor, which is applied to the X-rod 258 through the X-connector 262 causes controlled translational or linear movement of the X subsystem 250 in the X direction. The X-Y subsystem 204 also includes a Y subsystem 266 comprising a Y-frame 270, a Y-rod or track 274 and a Y-connector 278. The output from the Y servomotor communicates with the Y-rod 274 through the Y-connector 278 in connection with providing relative movement between the Y-rod 274 and the Y-frame 270 in order to enable movement of the Y subsystem 266 in the Y direction. The X subsystem 250 and the Y subsystem 266 are joined together using a X-Y plate 282 that is illustrated in FIG. 18.

The test piece subsystem 208 is joined to the X-Y subsystem 204 by, in this embodiment, portions of the Y-frame 270. The test piece subsystem 208 can include a test piece base 286, a test piece side 290 and a test piece front 294. As depicted in FIG. 17, each of these test piece parts can be joined together and the test piece 300 is held using these three test piece parts. The test piece base 286 is joined to the Y-frame 270 of the Y subsystem 266. Consequently, movement in the X direction and/or Y direction using the X-Y subsystem 204 causes movement of the test piece subsystem 208 including the test piece 300 having one or more samples that are to be analyzed by the instrument 200.

With regard to the laser subsystem 212, it is also joined to the base plate 304 to which the X-Y subsystem 204 is connected. Referring to FIG. 18, in one embodiment, the laser subsystem 212 includes the laser device 310 that is joined to a laser holder 314 which can be in the form of a C-clamp configuration having a cylindrical bore that receives the laser device 310. The laser holder 314 can have at least one slot 334. The laser holder 314 is joined to a laser support 318 having a foot portion 322 with a slit 326. The laser holder 314 can be held at a selected angular position to the laser support 318. Depending upon the location of the laser holder 314 relative to the slot 334, a selected, desired angle of the light beam output from the laser device 310 can be provided. The angle of the light beam is relative to the surface of the test piece 300. The laser support 318 is also joined to the base plate 304 and can be laterally, selectively positioned by joining the foot portion 322 to the base plate 304 at a selected part of the slit 326. Hence, the laser device 310 can be controllably positioned in a substantially lateral direction relative to the test piece subsystem 208 including the test piece 300 itself to obtain desired location of the laser light or light beam from the laser device 310 on the test piece 300.

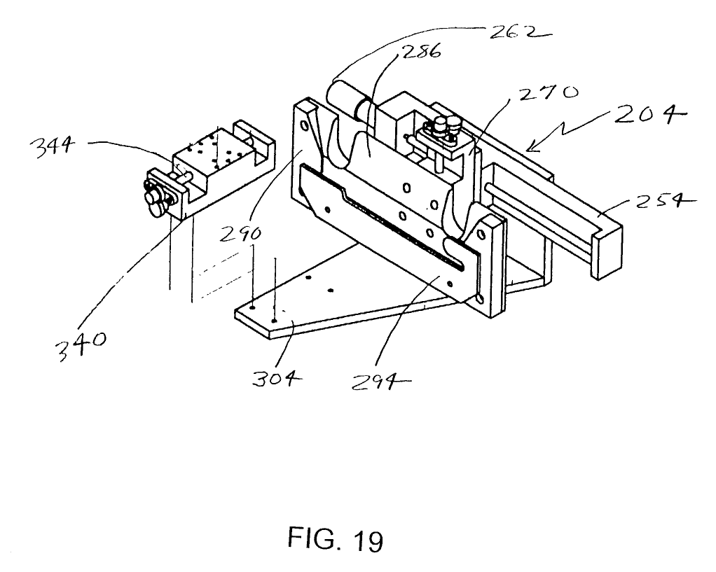

Referring to FIG. 19, a Z-rod or track 344 is joined to the Z-frame 340. The Z subsystem 232 can be manually movable whereby the Z-frame 340 moves relative to the Z-rod 344 to adjust its position in the Z direction relative to the test piece 300. In another embodiment, the Z subsystem 232 can be automatically controlled using the control 230.

Referring again to FIG. 18, a video objective 360 is illustrated that can be held by a lens cell holder 350 (FIG. 17). The lens cell holder 350 can also be a C-clamp configuration with a cylindrical bore that holds the video objective of the optical subsystem 216 used in receiving scattered light from the test piece 300. The embodiment of FIG. 18 also depicts a Z-plate 364 that is used to provide greater controlled movement in the Z direction. Attached to the Z-plate is a plate 368 to which the light collection device 220, such as the digital camera, can be joined in connection with achieving desired movement in the Z direction relative to the test piece 300.

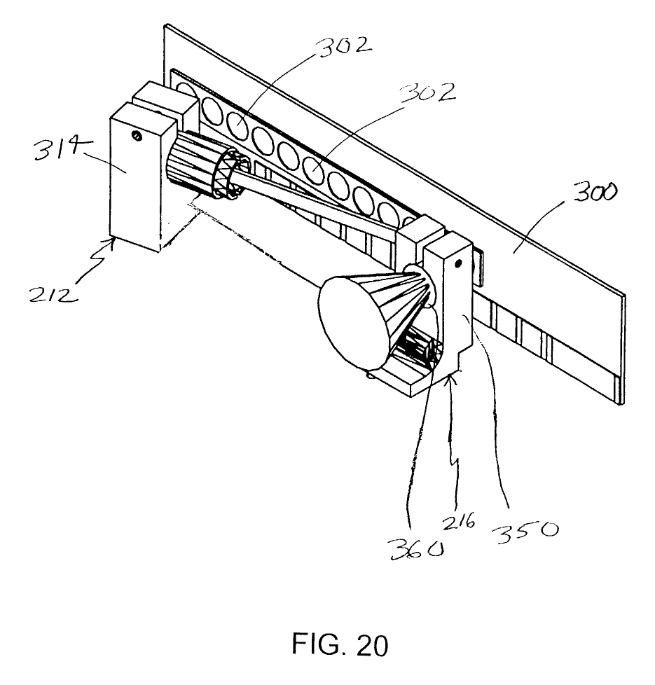

With reference to FIG. 20, a schematic representation is provided showing the light beam being output from the laser subsystem 212 to one of the test spots 302 on the test sample 300. The light beam is directed unobstructed to the subject test spot and from portions thereof, scattered light results. The scattered light is received by the optical subsystem 216 including its video objective 360. From there, the scattered light is directed to the light collection device 220 for subsequent processing. As can be understood, the laser subsystem 212 can be located at a desired angle relative to the test spots 302 by initial selective adjustment using the slot 334. The lens cell holder 350 can also be adjusted. In such a case, the adjustment is essentially linear in the Z direction. After completion of any such adjustment, the light beam is able to controllably strike or contact each of a selected or desired one of the spots 302. In particular, neither the optical subsystem 216 nor the light collection device 220 cause an obstruction to the light beam as it is directed to a particular spot 302 on the test piece 300. This unobstructed path remains as the test piece 300 is moved in X and Y directions during the relative movement between the test piece 300 and the light beam, as part of the testing of the test spots 302 in connection with determining whether a particular analyte or other substance of interest is present with one or more of the test spots 302.

In connection with the desired testing, the next description relates to certain controls and indicators that can be provided in achieving acceptable test results. FIG. 22 conveniently depicts a conglomeration of a number of software generated computer screens that relate to controllable functions useful in determining whether a particular substance of interest is present with the sample under test. Regarding the light collection device 220, such as a digital camera, each of its gain and its integration time (shutter speed) can be separately regulated. In one embodiment, a mouse or other input device to the computer of the control 230 is controlled by the operator or user in connection with increasing or decreasing one or both of the digital camera gain and shutter speed. Generally, the magnitudes of control for each of these two parameters of the light collection device 220 is determined by empirical information gathered or known by the operator. For example, in cases in which the substance of interest under test has been previously tested for, the information obtained concerning gain and shutter speed that achieved accurate or acceptable results in the previous test may be relied upon to determine whether that same substance of interest is present with the current sample being tested. The control of each of the gain and shutter speed is used to provide light or image data that enhances the acceptability or accuracy of the ultimate determination related to the detection and/or measurement of the substance of interest, if present. In one embodiment, the parameters of the light collection device 220 can be adjusted during processing/analyzing procedures in determining whether a substance of interest is present with the sample under test. The parameters can be initially provided and utilized during the testing and, subsequently, based on obtained information and processing/analysis that was completed, one or both of these parameters could be adjusted to better or enhance the image data being obtained. It is preferred that any such subsequent adjustment that might occur during testing be implemented automatically, which automatic determination can rely on one or more of a number of factors related to the intensity of the light being received.

The control panel 240 of FIG. 22 identifies a look up table (LUT) function or application, which can be selectively activated or de-activated by the operator using an input device, such as a “button” that can be controlled by touch, mouse manipulation or other suitable selection. When activated, the selected LUT application enhances the brightness and contrast of images (image data or other information) by modifying the dynamic intensity of image data or regions thereof that have relatively poor contrast. A LUT transformation converts input grey level values obtained by the light collection device 220 as a function of a sample under test into other grey level values that constitute a transformed image having transformed image data. The LUT applications that can result in such a transformation are essentially mathematical tools implemented by software that are executed by the computer of the control 230. There are a number of predetermined LUT applications for selection in connection with enhancing the brightness and contrast of the image data. These LUT applications can include the following: linear, log, exponential, square, square root, power X and power 1/X. One or more of these mathematical tools, or other similar tools, is selectable by the operator to achieve the desired function. Typically, if the LUT application is activated, only one of them is utilized for a particular test sample. As also seen in this illustration of the control panel 240, the operator can select a X value that is used when the LUT application is power X or power 1/X. The selected power is used with a pixel value and, in particular, mathematically manipulates or acts on that pixel value in conjunction with changing the dynamic range of the pixel values. The pixel refers to the smallest or finest dimension of the light collection device 220, such as the resolution of the digital camera that can be defined as including an array of pixels. In one embodiment, the pixel values can be in the range of 0-255, with a zero pixel value referring essentially to a black pixel and the pixel value 255 essentially referring to a completely white pixel. For example, the power X application is used to make particles, when present, appear bright on a uniformly black background. The value of X in this embodiment is about 2-3, such as 2.80. A mathematical calculation involves raising the pixel value to the 2.8 power in this example. For a pixel value of 100, the mathematical calculation involves 100^{2.8}. In accordance with this example, after the mathematical calculation relatively more pixels would be assigned a pixel value of 255 and other pixels would be assigned, on a relative basis, pixel values less than 255.

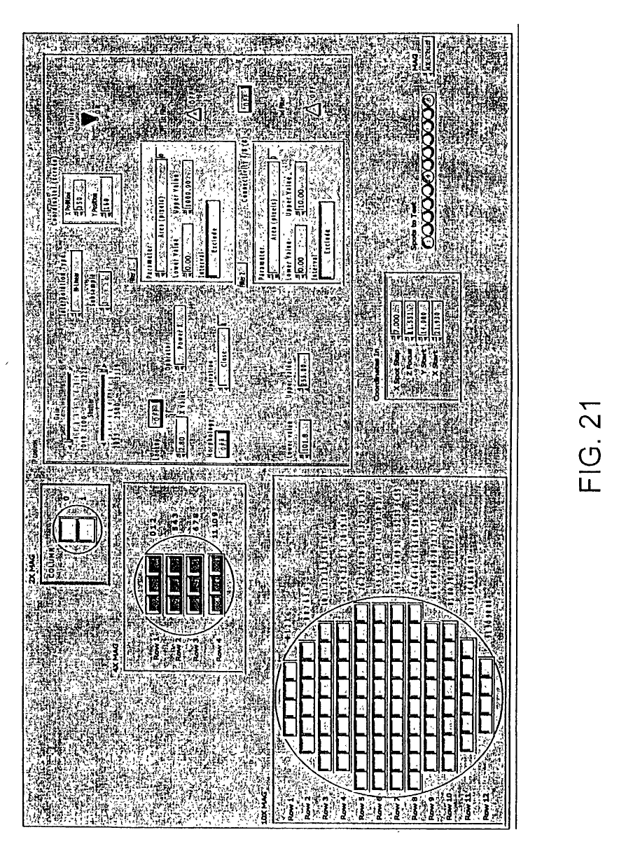

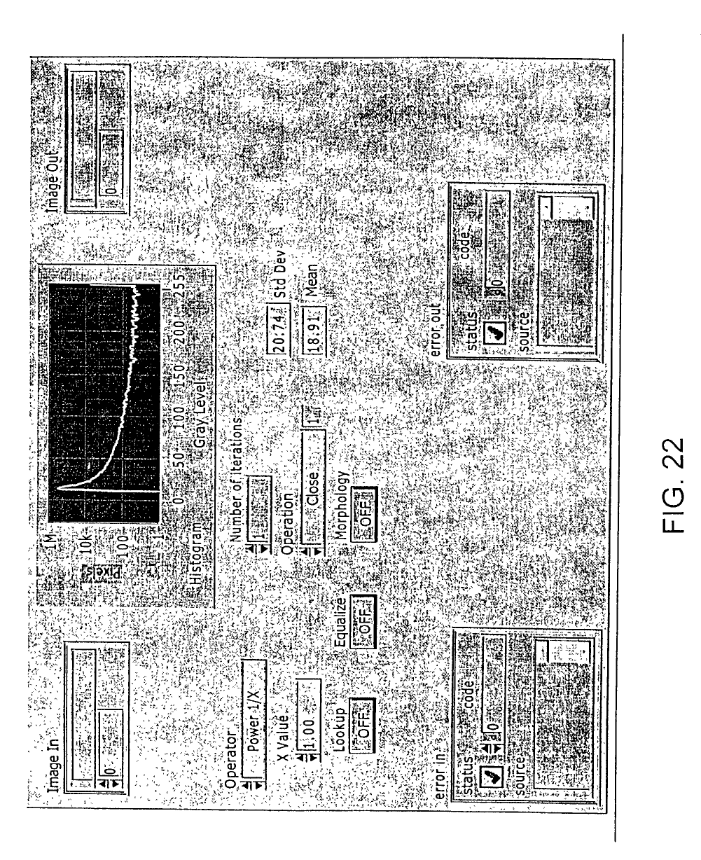

A thresholding control function is also identified by the control panel 240 of FIG. 21. Thresholding involves segmenting image data into two regions, namely, a particle (or object intended to be indicative of the target analyte) region and a background region. When implementing a thresholding process, all pixels can be set to a binary 1 when their pixel values equal or exceed a grey level value that can be defined as the lower limit threshold limit, while all other pixels having pixel values less than the lower limit threshold limit can be set to a binary 0. Alternatively, the pixels equal to or exceeding the threshold can be set to a binary 0 and those below can be set to a binary 1. In one embodiment, the lower limit threshold value, which is at the lower end of the thresholding interval, is determined using a histogram analysis. The histogram provides the frequency of a given distribution of pixel values for the particular collected image data. For example, if 100 pixels in the image data have a pixel value of 20, then the frequency for the pixel value of 20 is 100. Referring to FIG. 22, a representative histogram is illustrated for a grey level range of 0-255. The numbers of pixels are noted for different pixel values along the grey scale range. For each pixel value, an analysis is conducted using the number of pixels having that pixel value. A determination of the minimum threshold value or lower limit is determined by finding the maximum frequency peak for a given distribution of pixel values. Based on the determined minimum threshold value, any pixel value that is less than the minimum threshold value is assigned a binary 0 and those greater than or equal to the minimum threshold value are assigned a binary 1. As a result, those assigned a binary 0 are removed from any further consideration or analysis in connection with determining particles or objects evidencing the substance of interest. In one embodiment, a maximum value or upper limit can be defined and input to control which pixel values are to be used in the subsequent determinations. The maximum value or upper limit is typically operator selected and manually input using the mouse or other computer input device. In one embodiment, similar to gain or shutter speed settings, the maximum value is found empirically or by “trial and error.” Previous determinations of the upper limit for a particular substance of interest can be relied upon in arriving at the current maximum value. Referring to FIG. 21, the lower value and the upper value indicators refer to the minimum and maximum threshold values, respectively.

A further controllable function related to providing desired image data is the morphology function or application, which can also be activated or de-activated by the operator. Generally, the morphology function involves obtaining and altering the physical appearance or structure of particles in a binary image. The morphology function is typically utilized to enhance the image information in a binary image before making particle measurements related to their area, perimeter, and/or orientation, or other suitable particle measurement parameter. Since the morphology function relies on the binary image, it is usually conducted after the thresholding process. Because thresholding involves subjectivity, the resulting binary image may contain unwanted information, such as noise particles, particles touching a border of an image, particles touching each other, and particles with uneven borders. By affecting the shape of particles, the morphology function can remove such unwanted information and thereby provide better image data or information in the binary image.

In conducting a morphology function, one or more of a number of available tools or operations can be chosen by the operator. These can include the following, which are known and their meanings understood including their main objectives or functions in connection with such mathematical manipulations: auto median, close, dilate, erode, gradient, gradient in, gradient out, hit or miss, open, P close, P open, thick and thin. Regardless of which is utilized, each such function performs a pixel by pixel operation on the source binary image according to predefined functions. For example, the dilation function eliminates extremely small holes and islands in particles or objects and expands their contours accordingly. Another function that can be employed is the close function, which is an image processing tool that mathematically manipulates a particle that is almost a circle by closing it so that such particle becomes a complete circle or has a closed perimeter.

The control panel 240 also illustrates a filter1 function and a filter2 function. Each of these filter functions is also selectively controlled by the operator whereby one or both of filter1 and filter2 can be turned on/off. With respect to these filtering operations, information related to particle size can be obtained and particles falling within a given size related range may be counted or excluded from a particle or object count. In one embodiment, particle area is determined and particles are filtered based on that parameter. The area parameter can be defined in terms of the number of pixels. With respect to the filtering based on pixel area, one or more of a number of factors can be taken into account related to particle area. These include circumference, average diameter, area itself, minimum diameter, maximum diameter and aspect ratio (maximum to minimum diameter). With regard to qualifying or limiting the selected parameter, such as area, a lower value and/or upper value associated with the area can be input by the operator. One or both of these two values are also typically empirically determined. When using both filters, filter2 may be used to modify or perform a further filtering function based on the results of the filter 1 process. For example, prior to filtering, there may be particles of the same or essentially same area but having different shapes (e.g., one particle is similar to a rectangular shape while another particle is similar to a circular shape). Implementing filter1 based on area may result in remaining particles being identified that have essentially the same particle area but differ in shape. Filter2 may be employed to conduct a further filtering process by which only one of such two different shape particles are counted or taken into account in determining whether the substance of interest is present. The filter2 process may remove or filter out the rectangular shaped particle or the circular shaped particle, depending upon which parameter or factor is relied in performing the second filtering operation. This control factor may be based on a diameter or diagonal value that results in filtering or removing one of these two differently shaped particles, while the other remains for counting. In addition to filtering based on pixel area, other operations or mathematical tools can be employed including one or more of the following known and understood functions: mean chord Y, longest segment top row (Y), mean chord Y; max intercept, perimeter, max intercept; mean intercept perpendicular, holes perimeter, mean intercept perpendicular; particle orientation, sumX, particle, orientation; equivalent ellipse minor axis, sumY, equivalent ellipse minor axis; ellipse major axis, sumXX, ellipse major axis; ellipse minor axis, sumYY, ellipse minor axis; ratio of equivalent ellipse axis, sumXY, ratio of equivalent ellipse axis; rectangle big side, corrected projection X, rectangle big side; rectangle small side, corrected projection Y, rectangle small side; ratio of equivalent rectangle sides, moment of inertia 1xx, ratio of equivalent rectangle sides; elongation factor, moment of inertia 1yy, elongation factor; compactness factor, moment of inertia 1xy, compactness factor; Heywood circularity factor, mean chord X, Heywood circularity factor; type factor, mean chord Y, type factor; hydraulic radius, max intercept, hydraulic radius; Waddell disk diameter, mean intercept perpendicular, Waddel disk diameter; diagonal, particle orientation, diagonal.

A further function associated with analyzing particles or objects that might be utilized, is the connectivity function that relates to analyzing particles which are located diagonally adjacent to each other. In one embodiment, a connectivity factor of four or eight is available for use or selection by the operator. A connectivity of four means that such diagonal particles are counted as two distinct particles. A connectivity of eight means that the diagonally adjacent particles are recognized as one particle.

The control panel 240 of FIG. 21 also depicts operator control over interpolation of pixel values. An image data indicator related function is provided by means of the subsample indicator. According to this function, a correspondence or correlation is provided between the pixels associated with the digital camera and the pixels on the computer screen or display 234. When causing a display depicting the image data of the digital camera pixels, it may be desirable to have a reduced image size whereby a number of digital camera pixels corresponds to one point or pixel on the computer screen. For example, a subsample value of three means that the computer screen has one display point or one display pixel that corresponds to three digital camera pixels.

With respect to the test piece 300 and its test spots 302, the control panel 240 also has information related to the X, Y, and Z positioning thereto. These coordinates or values can be provided once for a particular test piece and then can be later used for other test pieces. However, if the coordinates should change, for example, the distance between test spots on the test piece is changed, then the X spot step value would need to be changed. The X spot step value indicates the distance between the centers of test spots on the test piece.

A display is also provided on the control panel 240 related to identifying the test piece spots that can be tested. In the embodiment illustrated, there are 12 test spots. The operator can control a particular test spot to be tested by selecting (e.g., using a mouse) one of the test spots to be tested and an indication is provided, such as by a color change or other identifier indicative of which test spot of the test piece is being tested or has been tested.

A magnification parameter is also identified on the control panel 240. As previously described, the instrument 200, particularly the optical subsystem 216, can be configured with or include different magnifications for selection. Since the selected magnification is a parameter used in the processing and analysis of image data, this magnification parameter is input to the control 230 so that the software can use that value in performing certain tasks. Related to the magnification parameter are graphic representations that can be provided using the display 240 related to the three possible embodiments of magnification, namely, 2×, 4× and 10×. With respect to each of these magnifications, a representation is provided of one test spot of the test piece that is to be tested. Depending upon the magnification, there are a different number of subspots. The greater magnification (10×) embodiment has a substantially greater number of subspots than the other two illustrated magnification embodiments.

When using the instrument 200, particularly the laser subsystem 212, the light beam covers and focuses on the entire spot so light strikes or is received by all test subspots of the test spot under test at the same time. Each subspot has a correlated or corresponding number of digital camera pixels. Thus, certain of predetermined pixels can be processed and analyzed for each particular subspot. Related to this arrangement is that different samples being tested could be provided on different subspots. That is, a first substance of interest might be tested using subspot one and a second substance of interest might be tested using subspot two. In determining whether one or more substances of interest is present with a test spot, each of the subspots can be separately processed and analyzed. As part of the enumeration method, the particles or objects that are counted after the image processing and analysis are completed can be separately counted for each subspot. In the case in which the same substance of interest is being tested for on all subspots of a particular test spot, after all the subspots have been analyzed and the particles counted for each, the total number of particles can be counted based on the counts made for each of the subspots. When each subspot or any number of subspots, which are less than all of the subspots for a particular test spot, have a first substance of interest, while one or more other subspots have at least a second substance of interest, separate particle counts are made for each such subspot or combination of subspots in determining whether a substance of interest is present. With respect to processing and analyzing subspots, in one embodiment, a substantially serpentine path is utilized when conducting such processing and analysis, particularly in an embodiment where there is a substantial number of subspots, such as the embodiment with the magnification of 10×. According to the serpentine path, the subspots of row 1 (0, 1, 2, 3) are separately analyzed in that order and then the subspots of row 2 (9, 8, 7, 6, 5, 4) are analyzed beginning with subspot 4. Then, for row 3 of subspots, the analysis is conducted right-to-left based on the representation in FIG. 22 and so forth until all subspots in row 12 have been processed and analyzed.

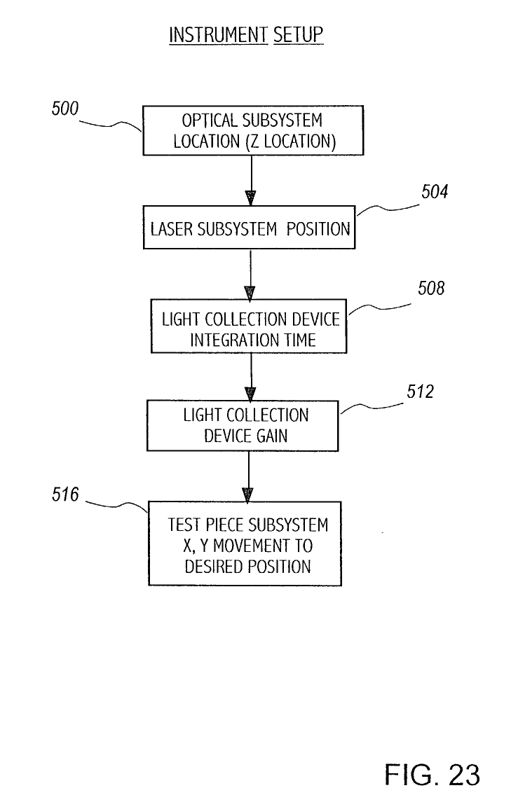

With reference to the flow diagrams of FIGS. 23-26, the operation of the instrument 200 is further described. Referring to FIG. 23, as part of testing one or more samples with a test piece 300, the operator or user initially establishes settings and/or positions associated with the instrument 200. At block 500, the optical subsystem 216, or one or more elements thereof, is located at a desired position in the Z direction. In the embodiment that includes FIG. 19, the objective tube lens can be positioned in the Z direction so that the optical subsystem 216 is desirably located relative to the test piece subsystem 208. According to one setup process, the optical subsystem 216 is located in an acceptable position and can remain in that position for any number of test piece subsystems 208 and samples being tested.

At block 504, steps can be taken to position the laser subsystem 212 so that its light beam output contacts or strikes the particular test spot 302 having the sample being tested without obstruction. Such positioning of the light beam can include adjustments related to lateral position and/or an angular position using the parts of FIG. 18. Like the setup for the optical subsystem 216, once it is finished for one sample being tested or one particular test piece subsystem 208, it may be that the laser subsystem 212 can remain in that position for any one of a number of samples being tested. The position of the laser subsystem 212 that affects the location of its light beam output can be automatically controlled, as well as manually controlled, just as can the location of the optical subsystem 216.

Settings for certain parameters of the light collection device 220, such as the digital camera, can be part of the instrument set up. At block 508, the integration time or shutter speed of the digital camera can be initially provided. Likewise, the gain of the digital camera can be initially established at block 512. Such initial settings for each of these two parameters can be based on previous tests or experiences related to the same or similar substance of interest being tested. The integration time and the gain can be set using the control panel 240 and an input device, such as a mouse. The integration time and the gain of the digital camera could also be automatically controllable including, for example, based on previous determinations of the values of these parameters for particular substances of interest that were tested.

At block 516, positioning of the test piece subsystem 208 having the test spots 302 is accomplished. In the case in which the test subspot to be tested is not properly located, the test piece subsystem 208 is moved using, for example, the X-Y subsystem 204 by means of the hardware or parts illustrated in FIGS. 17-19. In one embodiment, the indicator on the control panel 240 depicting the test spots available for testing for a particular test piece 300 having 12 test spots can be used to properly position the X-Y subsystem 204. Selecting a particular test spot using an input and the indicator on the control panel 240 can cause appropriate movement of the X-Y subsystem 204 so that there is proper alignment between the light beam and the selected test spot.

Once the appropriate setup of procedures or steps has been completed, and with the test piece subsystem 208 in place as well, testing of one or more test spots and/or test subspots can be conducted to obtain information regarding the presence of a substance of interest. With the laser of the laser subsystem 212 activated, at block 518 the light beam strikes the selected test spot such that uniform light covers at least the entire selected test spot and, preferably, greater than the entire test spot. After striking the test spot, scattered light is generated that is collected by the light collection device 220 at block 520. In one embodiment, the digital camera that includes a number of pixels collects the scattered light. In such an embodiment, one or more of a number of the pixels map to or correlate with particular portions or sections of the spot under test, such as subspots. At any instance in time, the digital camera can obtain information as a function of its integration time from one or more chosen pixels that might relate to portions, sections or subspots of the test spot under test. The collected light received by the pixels is converted to electrical signals. The electrical signals can be processed at block 530 to prepare the image data obtained from the collected light for determinations, particularly counting, related to the number of particles or objects that might be present and which are indicative of the substance of interest.

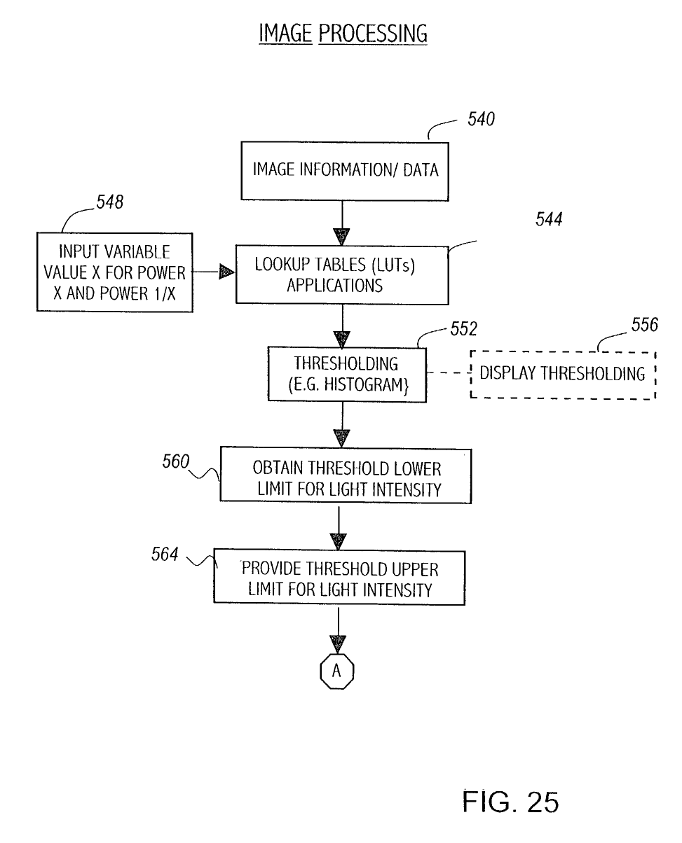

With reference to FIG. 25, main procedures or precesses available for processing the image data are illustrated. At block 540, the obtained image information/data is available for processing in the form of electrical signals and which information or data can be temporarily stored for processing using software and the algorithms that are executable using such software. At block 544, one or more look up tables (LUTs) can be accessed for manipulating the image data to enhance its brightness and/or contrast. That is, the image data obtained can be processed to provide a better representation thereof, such as desirably affecting the dynamic range of the obtained image data. In one embodiment, the available applications of LUTs include power X and power 1/X. When one of these application is to be used, at block 548 a value of X is input that is based on a desired or optimum contrasting or enhancement of the input image data.

Another imaging processing procedure that can be implemented is identified at block 552. In one embodiment, the thresholding procedure or function involves development of a histogram that is based on the pixel values currently received by the digital camera pixels. The frequencies of occurrence of such pixel values can be relied on in performing the thresholding. In one embodiment, at block 556, the results of the thresholding is displayed on the computer screen/monitor or display 234, such as in the form of a histogram or graph which displays the number of pixels having particular pixel values. At block 560, the lower limit related to light intensity is determined or obtained based on the thresholding. In one embodiment, the lower limit defines the boundary at which pixel values below it are assigned one binary value and pixel values at the lower limit and above are assigned the other binary value. In addition to the lower limit threshold, at block 564, an upper limit can also be provided related to light intensity. In one embodiment, the upper limit value is input by the operator, or has been previously stored and can be accessed for use. The upper limit value can be based on previous testing or other information that is relevant to its selection including operator knowledge or experience and other trial and error techniques. In another embodiment, the thresholding procedure may not be utilized. By way of example only, the gain and/or integration time associated with the digital camera may be suitably set so as not to require the thresholding function.

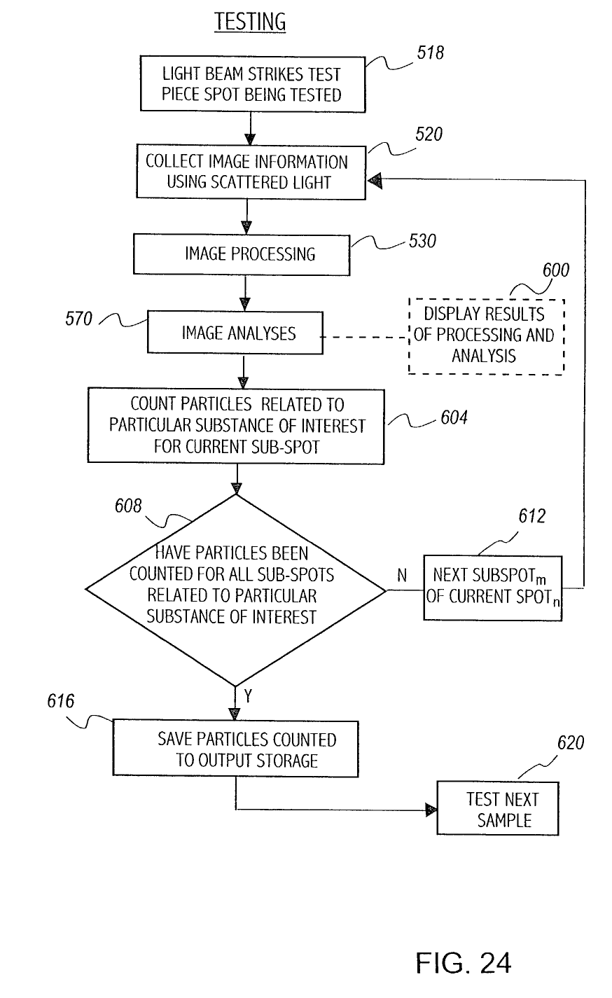

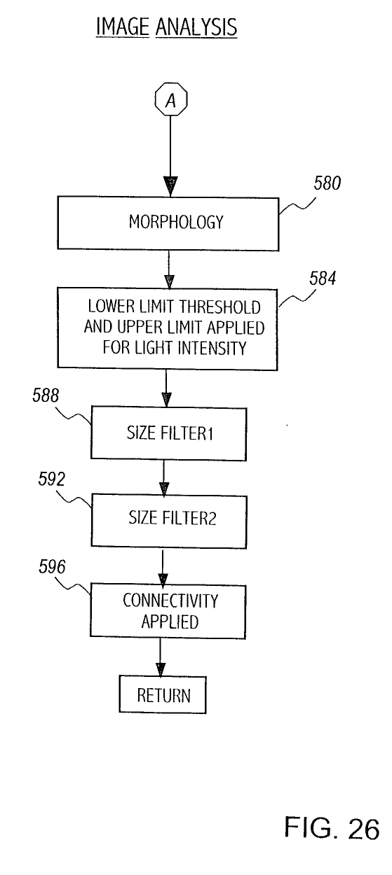

In addition to the availability for selection of image processing procedures, FIG. 24 identifies, at block 570 further procedures or series of steps that can be conducted as part of data image analyses. Referring to FIG. 26, such analyses can include one or more morphology procedures at block 580. In one embodiment, the morphology software can analyze the results of the image data after thresholding. One or more related but different morphology applications can be invoked related to the appearance or size of such image data. The morphology application can desirably manipulate the data (e.g. dilate and/or close functions) to better prepare it for more accurate counting of particles or objects when present that are indicative of the substance of interest.

At block 584, the resulting or current image data can have the lower limit threshold and upper limit applied thereto in connection with removing data or information that is deemed not to be relevant to or useful in the accurately determining the presence of the substance of interest.

Further procedures that can be implemented related to the size or appearance of image data involve one or more filtering functions. A block 588, size filter1 can remove or exclude certain image information based on input size parameters, as previously described. In one embodiment, at block 592, size filter2 can also be employed that, in one implementation, further filters the resulting image data after size filter1 has performed its function. At block 596, the connectivity feature could also be applied to essentially separate certain image data into more than one particle or object to be counted.

When one or more of these processees are completed and the determination is then to be made regarding the presence of the substance of interest based on the number of particles, a return is made to FIG. 24. In one embodiment, at block 600, the computer display or screen 234 can illustrate the result(s) of the processing and analysis that was conducted using the one or more procedures of FIGS. 25 and 26. Such results can include the number of particles that remain for counting or the counted number of particles that would be used in determining information related to the presence of the substance of interest. Regardless of whether or not such information is displayed, at block 604 of FIG. 24, based on the image data related to the particles that remain, the relevant software is used to count such particles or objects for the current subspot being tested. At decision block 608, a check is made regarding whether another subspot is to be tested in connection with determining the presence of the particular substance of interest. If there is one or more such subspots, at block 612, the next subspotₘ of the current spotₙ is next to be used in obtaining light information or image data therefrom. In that regard, the testing is repeated including a return to the series of steps associated with block 520. On the other hand, if all particles have been counted for a particular substance of interest, at block 616, the number of particles that have been counted for one or more subspots and/or spots being used to determine whether the substance of interest is present for a particular sample, is stored or saved to computer memory. If there is another sample to be tested, then at block 620, this further sample can be tested. In one embodiment, this next sample may be such that the previous instrument 200 set up need not be changed. If there is a need to change the instrument 200 setup, one or more of the procedures identified by the blocks of FIG. 23 can be employed before conducting the testing outlined by FIG. 24.

Although a number of techniques or procedures have been described related to processing and analyzing information related to whether a particular substance of interest is present, it should be appreciated that not all such procedures need be utilized for each test. Different combinations of processing and analysis could be employed. For example, it may be that no LUT is activated and the thresholding procedure is based on “raw” image data that is a function of the integration time and gain of the digital camera used in collecting the light information which defines the image data. In another example, there may be no upper limit associated with light intensity related to whether a pixel is a binary 1 or a binary 0; instead, only the lower limit threshold is used. In still another embodiment, only filter1 is used and not filter2 and not the connectivity application. The present invention provides substantial flexibility and diversity in conducting such processing and analysis. Generally, it is necessary to implement at least one processing/analysis feature or technique related to using light intensity of image data and one processing/analysis feature or technique related to using size or appearance of image data in order to best prepare the image data for counting of particles or objects. It should also be appreciated that changing one or more parameters and settings associated with the instrument 200 for a particular substance of interest that is being tested for, such as changing the magnification associated with the optical subsystem to 216, can cause or require other parameters or settings to change in order to achieve desired or accurate test results. For example, changing the magnification may require a change in the LUT application that is selected for use in enhancing the dynamic range of the image data. Regardless of any such change, the instrument 200 functions and allows operator control to respond to or adjust to such differences in order to make accurate determinations related whether or not the substance of interest is present.

The present invention also contemplates other parameters that might be useful in the enumeration method, such as obtaining or monitoring of a color factor that might be used in addition, or as an alternative, to processing and analysis related to light intensity and particle appearance or size. Other sources of light could be used instead of a laser beam. For example, multi-wavelength light could be used to strike the test spot.

It should also be understood that such obtaining of light information, together with processing and analysis thereof, is not limited to an embodiment in which the sample being tested moves relative to the light beam. The software implemented procedures and tools can also be used in embodiments in which the test piece is essentially stationary and the light beam is caused to move relative to the test piece. Relatedly, various combinations and permutations can be implemented as part of the present invention. In connection with controlling the position of the light beam, the source of the light (e.g. laser) could move. One or more of the light collection or detection components could move (e.g. optics, objective, light collector). The position of the light beam can also be controlled by movement of the test piece. Each of these components could move separately or together in order to desirably position the light beam on a test spot or a test sub-spot. Each movement of these parts or components could be accomplished by one or both of automatic control and manual control. Such part movements could be accomplished in one or more of a number of different directions, including laterally, longitudinally, angularly, pivotally, and/or rotationally. Furthermore, the light information could be obtained from an entire one test spot and a determination could be made as to whether the substance of interest is present with the one test spot. Light information could also be obtained individually from a number of test sub-spots. One or more of the test sub-spots could be used to determine the same or different substances of interest. The light information from the test spot or test sub-spots could also be processed essentially in real time or could be saved (stored in memory) for later, off-line analysis.

### EXAMPLE 1

Specific Binding Assay

Preparation of Whole Wafer Test Pieces. The test pieces used are commercially available 5′ silicon (Si) wafers. Thin layer polyurethane coated wafers are produced using standard spin-coating procedures to lay the polyurethane on the reflective surface of the wafer. Briefly, the wafers are prepared by addition of 500 μl of a thoroughly mixed 1.25% solution of Polymedica M1020 Polyurethane (Polymedica, Inc.) in N,N-dimethylacetamide (DMAC) (Sigma Chemical Co.) to the center of a silicon wafer (Addison Engineering) spinning at 5000 rpm. The wafer is air dried and then baked at 70° C. for 16-20 hours. Next, a 10 circle by 10 circle pattern is applied to the non-reflective wafer surface using a 3.5″×3.5″ rubber stamp coated with RTV 108 silicone rubber adhesive sealant (GE Silicones, Inc.). The resulting circular outlines serve as a means to isolate each circular polyurethane coated test spot (˜0.25″ diameter). The adhesive is cured at ambient room temperature for approximately 24 hours prior to use in assay.

Adsorption of Streptavidin Coated Microspheres to a Biotinylated Surface. Each of tilt the polyurethane coated wafer test spots are coated with 20 μl of a 1 μg/ml of biotinylated bovine serum-albumin (BSA) (Sigma Chemical Co.), or alternatively a non-biotinylated BSA for use as a negative control. The wafer is incubated at 37° C. for one hour in a 100% humidity chamber. After incubation, the wafers are rinsed 3 times with deionized water and dried with compressed air. Following BSA immobilization, the test spots are blocked with 30 μl of 3% BSA for 1 hour at 37° C., then rinsed 3 times with deionized water and dried with compressed air.

Streptavidin coated polystyrene microspheres (350 nm diameter) (Bangs Laboratories) are serially diluted in borate buffer (0.1 M, pH 8.5+0.01% Tween-20), for resulting dilution ranging between 1:10 and 1:10,000. Next, 20 μl of each dilution is applied to the biotinylated and non-biotinylated test spots and the wafer incubated at 37° C. for 1 hour, rinsed for 10 seconds with deionized water, compressed air dried and analyzed with the invention disclosed herein, the results of which are shown in Table I. These data show that light scattering labels bound to a surface can be detected and enumerated using the present invention; that streptavidin coated microspheres bind specifically to a biotinylated surface; and that the number of microspheres counted on the surfaces is dependent on the number applied to the surface.

### EXAMPLE 2

Staphylococcal Enterotoxin B (SEB) Detection Assgy

Preparation of Whole Wafer Test Pieces. The test pieces used are commercially available 5′ silicon (Si) wafers. Thin layer polyurethane coated wafers are produced using standard spin-coating procedures to lay the polyurethane on the reflective surface of the wafer. Briefly, the wafers are prepared by addition of 500 μl of a thoroughly mixed 1.25% solution of Polymedica M1020 Polyurethane (Polymedica, Inc.) in N,N-dimethylacetamide (DMAC) (Sigma Chemical Co.) to the center of a silicon wafer (Addison Engineering) spinning at 5000 rpm. The wafer is air dried and then baked at 70° C. for 16-20 hours. Next, a 10 circle by 10 circle pattern is applied to the non-reflective wafer surface using a 3.5″ on 3.5″ rubber stamp coated with RTV 108 silicone rubber adhesive sealant (GE Silicones, Inc.). The resulting circular outlines serve as a means to isolate each circular Polyurethane coated test spot (˜0.25″ diameter). The adhesive is cured at ambient room temperature for approximately 24 hours prior to test spot mounting on test piece and use in assay.

SEB Detection. A full sandwich assay is used for the detection of SEB in a sample buffer. The general protocol consists of coating capture antibody to individual test spots, blocking, adding different concentrations of SEB to the coated test spots, applying a bictinylated secondary reporting antibody, and labeling the bound secondary antibody with avidinated polystyrene microspheres.

Test wafers are coated with polyclonal ∝-SEB capture antibody by applying 20 μl of a 30 μg/ml (in 0.1 M PBS, pH 7.2) solution to each assay test spot. The wafer is incubated at 37° C. for 1 hour to allow passive adsorption of the capture antibody to the polyurethane. After incubation, the wafer is rinsed 3 time with deionized water and dried with compressed air.

Following capture antibody immobilization, each test spot is blocked with 40 μl of a 3% BSA solution (0.1 M PBS, pH 7.2) to reduce nonspecific protein adsorption from subsequent assay steps. The wafer is incubated at 37° C. for 1 hour and subsequently. rinsed 3 times with deionized water and dried with compressed air.

SEB samples are prepared by serial dilution of a 1 mg/ml stock into sample buffer (0.1 M PBS+1% BSA+0.01% Tween-2-, pH 7.2), with final toxin concentrations ranging from 0.1 ng/ml to 100 mg/ml. Buffer with no SEB is used as a negative control. Twenty μl of each of the dilutions and the negative control are applied to separate test spots across the wafer surface. The water is incubated at 37° C. for 30 minutes then rinsed 3 times with deionized water and dried with compressed air.

Biotinylated ∝-SEB antibody is diluted to 4 μg/ml in sample buffer. Each test spot is coated with 20 μl of this secondary antibody dilution. The wafer is incubated at 37° C. for 30 minutes then rinsed 3 times with deionized water and dried with compressed air.

Test spots are coated with 20 μl of a 1:100 dilution of streptavidin coated 350 nm diameter polystyrene microspheres in borate buffer (0.1 M, pH 8.5+0.01% Tween-20). The wafer is incubated at 37° C. for 30 minutes then each test spot is rinsed for 10 seconds, dried with compressed air and analyzed. The results of such analysis are shown in Table II. These data show that the present invention can be used to enumerate the binding of an antigen to a solid phase in a specific and quantitative manner. The lower limit of detection for this method is 550 μg/ml.

Data acquisition and analysis are performed as generally described herein. The wafer or test piece is mounted on a stage, positioned, focussed and images captured. Data analysis includes employing a macro program within Image Pro Plus.

While the above description contains many specificities, these specificities should not be construed as limitations on the scope of the invention, but rather exemplification of the preferred embodiment thereof. That is to say, the foregoing description of the invention is exemplary for purposes of illustration and explanation. Without departing from the spirit and scope of this invention, one skilled in the are can make various changes and modifications to the invention to adapt it to various usages and conditions. As such, these changes and modifications are properly, equitably, and intended to be within the full range of equivalence of the claims. Thus, the scope of the invention should be determined by the appended claims and their legal equivalents, rather than by the examples provided herein.

TABLE I
Specific Adsorption of Beads to Biotinylated Surfaces

| | # Objects: | # Object: |
|---|---|---|
| Bead Dilution | Biotinylated Surface | non-Biotinylated Surface |
| 0.0486111111 | 2263 | 201 |
| 1:100 | 2019 | 27 |
| 1:500 | 1375 | 9 |
| 1:1000 | 849 | 13 |
| 1:10,000 | 115 | 8 |

TABLE II
SEB Detection Assay

| SEB Concentration | | |
|---|---|---|
| (ng/ml) | # Objects | Standard Deviation |
| 0 | 62 | 5 |
| 0.1 | 72 | 12 |
| 0.5 | 121 | 10 |
| 1 | 203 | 51 |
| 10 | 906 | 281 |
| 100 | 1800 | 353 |

what is claimed is:

1. A method for analyzing a sample for binding events when a substance of interest is present with the sample, comprising:
establishing at least one of a setting and a position for an instrument that includes a control and that is involved with making determinations related to at least the presence of the substance of interest with the sample;
positioning the sample relative to a light source that outputs a light beam;
receiving said light beam by at least portions of the sample;
collecting scattered light from the sample portions using a light collection device of said instrument;
processing digital image data based on said light collected during said collecting step using said control of said instrument; and
counting objects after said processing step using digital information in determining at least whether the substance of interest is present with the sample.

2. A method, as claimed in claim 1, wherein:
said establishing step includes providing magnification related to collecting said scattered light.

3. A method, as claimed in claim 1, wherein:
said establishing step includes locating an optical subsystem in a direction relative to the sample.

4. A method, as claimed in claim 1, wherein:
said establishing step includes locating said light source such that said light beam is at a desired angle relative to the sample.

5. A method, as claimed in claim 1, wherein:
said collection device includes a photoelectric device and said establishing step includes regulating at least one of integration time and gain related to said photoelectric device to provide desired light contrast.

6. A method, as claimed in claim 1, wherein:
said light beam includes a laser beam and the sample is associated with a test spot and said establishing step includes having said laser beam encompass at least all of said test spot with uniform light intensity.

7. A method, as claimed in claim 1, wherein:
said positioning step includes moving at least one of the sample and said light beam.

8. A method, as claimed in claim 1, wherein:
said processing step includes receiving electrical signals from said light collection device and obtaining said image data using said electrical signals.

9. A method, as claimed in claim 1, wherein:
said processing step includes using at least a first light intensity related procedure and at least a first size related procedure.

10. A method, as claimed in claim 9, wherein:
said first light intensity related procedure includes at least one of: enhancing a dynamic range related to light intensity; implementing at least one lookup table application related to light contrasting; performing a thresholding function related to light intensity; and utilizing a lower limit threshold based on pixel values associated with said light collection device.

11. A method, as claimed in claim 10, wherein:
said performing step includes using a histogram analysis.

12. A method, as claimed in claim 9, wherein:
said first size related procedure includes at least one of: conducting a morphology application; filtering using at least one parameter related to size; and performing a connectivity function related to adjacent objects.

13. A method, as claimed in claim 1, wherein:
said processing step includes providing a lower limit threshold based on histogram-related information.

14. A method, as claimed in claim 1, further including:
storing information in memory of said control related to said at least one of said setting and said position.

15. A method, as claimed in claim 1, further including:
adjusting at least one of integration time and gain associated with said light collection device after conducting at least some of said processing step.

16. A method, as claimed in claim 1, wherein:
the sample includes a test spot comprised of at least a first subspot and a second subspot immediately adjacent to said first subspot and in which said processing step includes obtaining said image data using said collection device from said first subspot, and separately obtaining said image data from said second subspot, and said counting step includes counting objects from said first subspot before obtaining said image data from said second subspot.

17. A method, as claimed in claim 1, wherein:
the substance of interest is a first substance of interest and said image data from said first subspot includes information related to the first substance of interest when present and said second subspot has a second sample, different from the first sample, to be used in determining whether a second substance of interest, different from the first substance of interest, is present.

18. A method, as claimed in claim 1, wherein:
said digital image data is based on a two dimensional array of elements.

19. A method, as claimed in claim 1, wherein:
the sample has a light-scattering label that includes at least one of: colloidal gold, selenium, silica particles, magnetic particles, metal particles, metal coated particles and polymer particles and in which said polymer particles are made of at least one of: latex, polystyrene, polymethylacrylate and polycarbonate.

20. An apparatus for counting binding events associated with a substance of interest when present with a sample, comprising:
a test subsystem that holds at least a first sample;
a light subsystem that outputs a light beam directed to the first sample;
an optical subsystem that receives scattered light from the first sample;
a light collection device in communication with said optical subsystem that collects scattered light; and
a control that processes digital image data related to the scattered light collected by said light collection device, said control using processed data to identify binding events in determining whether the substance of interest is present with the first sample.

21. An apparatus, as claimed in claim 20, wherein:
said control conducts at least a first procedure related to light intensity and conducts at least a second procedure related to size to provide said processed data.

22. An apparatus, as claimed in claim 20, wherein:
said processed data includes first processed data obtained by said control executing software that enhances light contrast of said image data based on said light intensity.

23. An apparatus, as claimed in claim 20, wherein:
said processed data includes second processed data obtained by said control executing software for use in obtaining a lower limit threshold related to said light intensity.

24. An apparatus, as claimed in claim 20, wherein:
said processed data includes third processed data obtained by said control executing software that filters said image data based on information related to size.

25. An apparatus, as claimed in claim 20, wherein:
said processed data includes fourth processed data that enhances differences related to said image data based on said light intensity.

26. An apparatus, as claimed in claim 20, wherein:
said processed data includes a lower limit threshold that is obtained by said control executing software related to a histogram based on pixel values.

27. An apparatus, as claimed in claim 20, wherein:
said processed data includes a plurality of magnitudes related to said light intensity that are at least equal to or greater than a lower limit threshold.

28. An apparatus, as claimed in claim 20, wherein:
said light collection device includes a photoelectric device having a first setting related to integration time and a second setting related to gain.

29. An apparatus, as claimed in claim 20, wherein:
said test subsystem is movable in a X direction and a Y direction using said control and a X-Y subsystem, the sample includes a plurality of test spots including a first test spot and a second test spot, said first test spot being defined to have at least a first subspot and a second subspot and in which said control and said X-Y subsystem moves said test subsystem to enable said light beam to be directed to said second spot.

30. An apparatus, as claimed in claim 20, wherein:
said light beam is caused to move in a X direction and a Y direction using said control.

31. An apparatus, as claimed in claim 20, wherein:
said control includes a memory for storing at least one of the following positions and settings associated with the apparatus: a magnification associated with said optical subsystem; a position of said optical subsystem in a Z direction, an angle related to said laser subsystem; an integration time of said light collection device; and a gain of said light collection device.

32. An apparatus, as claimed in claim 20, wherein:
said control includes a display that displays histogram-related information.
